(19) United States
(12) Patent Application Publication
(10) Pub. No.: US 2023/0040639 A1
(43) Pub. Date: Feb. 9, 2023

(54) TGFbeta1 Hyperactivation Causes Gender-Specific Calcific Aortic Stenosis

(71) Applicant: University of South Carolina, Columbia, SC (US)

(72) Inventor: Mohamad Azhar, lrmo, SC (US)

(73) Assignee: University of South Carolina, Columbia, SC (US)

(21) Appl. No.: 17/783,381

(22) PCT Filed: Oct. 30, 2020
(86) PCT No.: PCT/US2020/058148

§ 371 Date: Jun. 8, 2022

Related U.S. Application Data
(60) Provisional application No. 62/954,884, filed on Dec. 30, 2019.

(51) Int. Cl. A01K 67/027

(52) U.S. Cl. CPC A01K 67/0278; A01K 2217/052; A01K 2217/15; A01K 2217/206; A01K 2227/105; A01K 2267/0375

(57) ABSTRACT

The current disclosure has identified novel developmental, cellular, molecular, and biochemical pathways and developed a unique mouse model which recapitulates age, bicuspid aortic valve-associated, and gender-specific pathological aspects of development and progression of human CAVD, which will be useful in developing novel diagnostic, preventative, and therapeutic strategies for CAVD patients.

### STATEMENT REGARDING FEDERALLY SPONSORED RESEARCH

This invention was made with government support under RO1 HL126705 and and R01CA218578 awarded by the National Institutes of Health. The government may have certain rights in the disclosure.

### 1) TECHNICAL FIELD

The subject matter disclosed herein is generally directed to novel developmental, cellular, molecular, and biochemical pathways and developed a unique mouse model which recapitulates age, bicuspid aortic valve-associated, and gender-specific pathological aspects of development and progression of human CAVD, which will be useful in developing novel diagnostic, preventative, and therapeutic strategies for CAVD patients.

### 2) BACKGROUND

Calcific aortic valve disease (CAVD) affects 25% of the population over 65 years of age. CAVD is a progressive disease ranging from aortic valve (AoV) sclerosis to AoV stenosis (AS). CAVD is the third most common cardiovascular disorders after hypertension and coronary vascular diseases. It effects more than 5 million people in the US and is the leading cause of valve replacement surgery with 80,000 AVR/year in the U.S. FIG. 1 shows the progression of CAVD in an aortic valve. FIG. 2 shows the asymmetric nature of CAVD and illustrates the presence of fibrotic material in the valve body of a normal aortic valve and a calcified nodule at the base of an aortic valve with AS.

In the first phase of the disease, termed aortic sclerosis, the valve becomes thickened and mildly calcified but these changes do not cause any obstruction to blood flow. Over the years, the disease evolves to severe valve calcification and thickening with impaired leaflet motion and significant blood flow obstruction, which are hallmarks of calcific AS.

In developed countries, AS has a prevalence of 0.4% in the general population and 1.7% in the population greater than 65 years old. Congenital abnormality (bicuspid aortic valve with predominantly fused left (L) and right (R) coronary cusps), male gender, and older age are powerful risk factors for calcific AS.

There is no medical treatment for CAVD, and without surgery, AS leads to death. Currently, aortic valve replacement (AVR) remains the only effective treatment for severe calcific AS. The developmental, cellular, molecular, and biochemical mechanisms involved in development and progression of CAVD remain poorly understood.

Previous studies had shown that the multifunctional cytokine transforming growth factor beta1 (TGFB1) levels are increased in surgical specimens of the aortic valve leaflets obtained from patients with clinical aortic stenosis.

Calcific aortic valve stenosis is the third leading cause of heart disease and its incidence is increasing as the general age of the population increases. There is no medical treatment. Accordingly, it is an object of the present invention to novel diagnostic, preventative, and therapeutic strategies for CAVD patients based on a novel mouse model. The current disclosure has identified novel developmental, cellular, molecular, and biochemical pathways and developed a unique mouse model which recapitulates age, bicuspid aortic valve-associated, and gender-specific pathological aspects of development and progression of human CAVD. Thus, the findings of the current disclosure will be useful in developing novel diagnostic, preventative, and therapeutic strategies for CAVD patients.

Citation or identification of any document in this application is not an admission that such a document is available as prior art to the present disclosure.

### SUMMARY

The above objectives are accomplished according to the present disclosure by providing a mouse model. The mouse model may include at least one transgenic Tgƒb1^{TG};PostnCre mouse bred from a Tgƒb1^{Tg} female mouse and a Peri CreTg,+male mouse and the at least one transgenic Tgƒb1^{TG};PostnCre mouse may overexpress bioactive TGFβ1. Further, the mouse model may not require any additional experimental insults including hyperlipidemia, hypercholesterolemia, hypertension, diabetes and/or dietary supplements for inducing carotid artery stenosis. Still yet, Tgƒb1 cDNA of the Tgƒb1^{TG};PostnCre mouse may have mutated to prevent assembly of at least one latent associated peptide. Yet again, the at least one transgenic Tgƒb1^{TG};PostnCre mouse may be born with three aortic valve leaflets wherein right and left coronary cusps of an aortic valve fuse at a base and form a raphe as the at least one Tgƒb1^{TG};PostnCre mouse grows. Further yet, fusing of the right and left coronary cusps may be an acquired phenotype and not a congenital malformation. Again, mouse genetic sequence 1 may be changed to mouse genetic sequence 2 via changing Cysteine at 223 and 225 to Serine. Further again, calcific AS caused by GFβ1 hyperactivation predominantly in the at least one transgenic Tgƒb1^{TG};PostnCre mouse may be mediated by increased activation of SMAD2, SMAD3, SMAD15/9 and p38 MAPK; and decreased activation of pERK1/2 MAPK signaling pathways. Moreover, exposing the at least one transgenic Tgƒb1^{TG};PostnCre mouse to TGFβ receptor I kinase (S13431542) inhibitor may attenuate initiation of CAVD in utero. Further yet, CAS may show sex-specific disparity with male transgenic Tgƒb1^{TG};PostnCre mice developing CAS. Again yet, CAS may be asymmetric in initiation and progression of calcification in the at least one transgenic Tgƒb1^{TG};PostnCre mouse.

In a further embodiment, a method may be provided for creating a mouse model. The method may include mating at least one Tgƒb1^{Tg} female mouse with at least one Peri CreTg,+male mouse to produce at least one transgenic Tgƒb1^{TG};PostnCre mouse that overexpresses bioactive TGFβ1. Still yet, the method may include mating at least one Tgƒb1+/−female mouse to a Tgƒb1^{Tg},+; Peri CreTg,+male mouse to produce at least one Tgƒb1^{Tg},+; Peri CreTg,+;Tgƒb1+/−mouse. Further, the method may not require any additional experimental insults including hyperlipidemia, hypercholesterolemia, hypertension, diabetes and/or dietary supplements for inducing carotid artery stenosis in the at least one transgenic Tgƒb1^{TG};PostnCre mouse. Again, the method might include mutating Tgƒb1 cDNA of the Tgƒb1^{TG};PostnCre mouse to prevent assembly of at least one latent associated peptide. Still yet, the method may produce at least one male transgenic Tgƒb1^{TG};PostnCre mouse with three aortic valve leaflets wherein right and left coronary cusps of an aortic valve fuse at a base and form a raphe as the at least one male Tgƒb1^{TG};PostnCre mouse grows. Yet further, fusing of the right and left coronary cusps is an acquired phenotype and not a congenital malformation. Further again, the method may include mutating mouse genetic sequence 1 to mouse genetic sequence 2 via changing Cysteine at 223 and 225 to Serine. Still yet, the method may include causing calcific AS by GFβ1 hyperactivation predominantly in the at least one transgenic Tgƒb1^{TG};PostnCre mouse mediated by increased activation of SMAD2, SMAD3, SMAD1/5/9 and p38 MAPK; and decreased activation of pERK1/2 MAPK signaling pathways. Again moreover, the method may include exposing at least one transgenic Tgƒb1^{TG};Postncre mouse to TGFβ receptor I kinase (SB431542) inhibitor in utero to attenuate initiation of CAVD. Further still again, CAS may show sex-specific disparity with male transgenic Tgƒb1^{TG};PostnCre mice developing CAS.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows the progression of Calcific Aortic Stenosis in an aortic valve.

FIG. 2 shows the asymmetric nature of CAVD in an aortic valve.

FIG. 3 shows transforming growth factor beta1 (TGFB1) is increased in human calcific aortic stenosis.

FIG. 4A shows generation of endocardial cushion cell lineage-specific Tgƒb1 transgenic mice.

FIGS. 4B and 4C show increased levels of hemagglutinin (HA) epitope tagged constitutively active form of TGFβ1 in adult AoV.

FIGS. 4D and 4E show echocardiography showing signs of aortic stenosis in Tgƒb1^{Tg} mice (12-months).

FIGS. 4F, 4G, 4H, 4I, and 4M show H&E showing aortic stenosis and cartilage formation in thick AoV leaflets in Tgƒb1^{Tg} mice (12-mo).

FIGS. 4J, 4K, 4L, 4M and 4Q show Alizarin red staining showing AoV calcification in Tgƒb1^{Tg} mice (12-mo).

FIGS. 4N and 4M also show HA Tag IHC showing PostnCre-expressing expressing VIC containing bioactive HA-tagged TGFβ1 in Tgƒb1^{Tg} mice (12-mo) with aortic stenosis.

FIGS. 5A-5U increased TGFβ1 in VIC leads to fibrocalcific remodeling and calcific aortic stenosis in 12-mo-old mice.

FIGS. 5A, 5B, 5C, 5D, 5E, and 5F show pentrachrome comparisons of Control and Tgƒb1^{Tg}; PostnCre.

FIGS. 5G, 5H, 5L and 5J show Alcian Blue comparisons of Control and Tgƒb1^{Tg}; PostnCre.

FIGS. 5K and 5L show von Kossa comparisons of Control and Tgƒb1^{Tg}; PostnCre.

FIGS. 5M, 5N, 50, and 5P show Trichrome comparisons of Control and Tgƒb1^{Tg}; PostnCre.

FIG. 5Q shows TGFβ pathway gene expression.

FIG. 5R shows chondorgenic differentiation.

FIG. 5S shows osteogenic differentiation.

FIG. 5T show a BMP pathway.

FIG. 5U shows VIC activation.

FIGS. 6A-6M show TGFβ1 hyper-activation suppresses outflow tract cushion mesenchymal apoptosis and induces osteo-chondrogenic differentiation during heart development.

FIGS. 6A. 6B, 6C, and 6D show increased cartilage-specific matrix (Aggrecan) and myofibroblasts-specific protein (POSTN) in Tgƒb1^{Tg} mice (12-mo).

FIGS. 6E and 6F show AoV calcification is seen as early as 5-7 weeks old. Tgƒb1^{Tg} mice.

FIGS. 6G, 6H, 6I, and 6J show ME showing mesenchymal condensation of RV OFT cushion mesenchyme in Tgƒb1^{Tg} embryos (e13-14).

FIGS. 6K, 6I, and 6M show TUNEL, showing decreased cushion mesenchymal cell apoptosis in Tgƒb1^{Tg} embryos (e13.5).

FIGS. 7A-7P show TGFβ1 hyper-activation causes increased augmentation and osteo-chondrogenic differentiation of cardiac neural crest derived cushion mesenchyme.

FIGS. 7A, 7B, 7C, 7D, 7E, and 7F show histological staining showing ectopic cartilage formation in NC-specific Tgƒb1^{Tg} fetuses (P1).

FIGS. 7G, 7H, 7I, 7J, 7K, and 7L show histological staining showing ectopic cartilage formation in NC-specific Tgƒb1^{Tg} fetuses (P1).

FIGS. 7M, 7N, 70, and 7P show LacZ staining of cultured hearts ex vivo showing that inhibition of TGFβ signaling (via TGFβR1) can block enhanced NC augmentation in NC-Tgƒb1^{Tg} embryos.

FIGS. 8A-8L show dysregulated NAD-dependent HDAC enzyme (Sirtuin1) and poly-ADP(ribose) polymerase (PARP1) contributes to calcific aortic stenosis.

FIGS. 8A, 8B, 8C, 8D, 8E, 8F, 8G, 8H, and 8I show biochemical analyses showing increased SIRT1 and decreased NAD and PARP1 in 10-mo-old Tgƒb1^{Tg} mice.

FIGS. 8J, 8K, and 8L show Alcian blue showing that inhibition of SIRT1 can block CAVD in Tgƒb1^{Tg} mice (7-weeks).

FIGS. 9A and 9B show western blotting showing increased activation of SMAD2. SMAD1/5/9, and p38-MAPK (noncanonical signaling) in young and old Tgƒb1^{Tg} mice.

FIGS. 9C, 9D, 9E, 9F, 9G. 9H, and 9I show Aldan blue showing that TGFβ-neutralizing antibodies and Tgƒb1-haploinsufficiency and TGFβR1 signaling inhibition (via SB-431542) can block CAVD in Tgƒb1^{Tg} mice (7-weeks).

FIG. 10 shows a depiction of the mechanism of TGFβ signaling in the calcific aortic stenosis.

FIG. 11A shows at generation of VIC-specific Tgƒb1 transgenic mice.

FIG. 11B shows Western blot analysis of dissected AV from 15-month-old mice showing cleaved, mature peptide (m) and unprocessed, pro-peptide (p) from endogenous TGFβ1 and transgenic hemagglutinin (HA) epitope tagged constitutively active form of TGFβ1.

FIG. 11C show Alizarin red staining of heart cross sections from control (left panel).

FIG. 11D shows micro-CT scans of the heart to evaluate the extent of AV calcification were obtained from 15-20-months-old mice.

FIG. 11E shows two independent Tgƒb1^{TG};PostnCre (middle and right panels) mice to evaluate AV calcification.

FIG. 11F also shows micro-CT scans of the heart to evaluate the extent of AV calcification were obtained from 15-20-months-old mice.

FIG. 11G shows Doppler display of peak velocity across the normal and stenotic aortic valve was measured at 1.5 m/s for control (g, top) and 3.5 m/s 585 for transgenic mice, respectively.

FIG. 11H shows color Doppler interrogation showing a mosaic-color jet at the aortic valve during systole in transgenic mice (highlighted by an arrow).

FIG. 11I shows quantification of total TGFβ1.

FIG. 11J shows quantification of ectopic AV calcification from micro-CT images.

FIG. 11K shows quantification of AV peak velocity.

FIG. 11L shows quantification of AV area.

FIG. 12A shows H&E staining of heart cross sections of control male (left panel) and Tgƒb1^{TG};PostnCre (2 and 3 are magnified images of 1) male mice showing the fusion (arrow, image 1) of right (rc1) and left (1c1) coronary leaflets.

FIG. 12B shows Russel-Movat Pentachrome staining of heart cross sections.

FIG. 12C shows Von Kossa and Russel-Movat pentachrome staining of heart cross sections from control and Tgƒb1^{TG};PostnCre mice to illustrate AV calcification and cartilage formation at 2 months of age.

FIG. 12D shows representative cross-sectional multiphoton images of control and Tgƒb1^{TG};PostnCre mice at 12-months of age.

FIG. 12E shows H&E staining to show intra-cardiac condensed mesenchymal cellular nodules in the AV-hinge region (arrows, magnified image of the boxed region) in the Tgƒb1^{TG};PostnCre embryos (E14.5)

FIG. 12F shows analysis of gene expression markers in microdissected tissue from the AV region by qPCR in 4-month-old control and Tgƒb1^{TG};PostnCre mice.

FIG. 12G shows analysis of gene expression markers organized around cartilage differentiation.

FIG. 12H shows analysis of gene expression markers organized around osteoblast differentiation

FIG. 12I shows analysis of gene expression markers organized around BMP signaling pathway

FIG. 12J shows analysis of gene expression markers organized around valve interstitial cells activation or myofibroblast differentiation (α-smooth muscle actin (Acta1), collagen 1al, periostin (Postn)).

FIG. 13 shows sex-specific differences in TGFβ signaling underlies the pathogenesis of calcific aortic valve stenosis at: (a) Western blots of 14-month-old control female (n=3), control male (n=3), Tgƒb1^{TG};PostnCre female (n=3) and Tgƒb1^{TG};PostnCre male (n=3) showing changes of phosphorylated and total proteins in canonical (pSMAD2/SMAD2, pSMAD3/SMAD3, pSMAD1/5/9/SMAD1/5) and non-canonical (pp38 MAPK/p38 MAPK) TGFβ signaling pathways in microdissected AV tissue. β-actin was used as a loading control. Data are mean±s.e.m. P-values and individual values are indicated. Unpaired Two-sided Student's t-tests (without corrections for multiple comparisons) were used.

FIG. 14A shows at: (a) Western blot of control and Tgƒb1^{TG};PostnCre mice showing post-translation modification (acetylation, ADP ribosylation) in the microdissected AV tissue; and at (b) total NAD+ content in microdissected AV tissue samples from control and Tgƒb1 ^{TG};PostnCre mice.

FIG. 14B shows Western blots of control and Tgƒb1^{TG};PostnCre male mice.

FIG. 14C also shows Western blots of control and Tgƒb1^{TG};PostnCre male mice.

FIG. 14D shows Alizarin red staining of heart cross sections from 24-month-old control (left panel), 2 independent Tgƒb1^{TG};PostnCre (1 middle panels), and Tgƒb1^{TG};PostnCre;Tgƒb1^{+/Flox}(2 right panels) mice to evaluate AV calcification.

FIG. 14E shows models illustrating the TGFβ1-dependent molecular pathways involved in AV calcification in females and calcific AS in males.

FIG. 14F also shows models illustrating the TGFβ1-dependent molecular pathways involved in AV calcification in females and calcific AS in males.

FIG. 15 shows analysis of AV leaflets fusion in male VIC-Tgƒ1^{TG} mice by 3 D morphological reconstruction.

FIG. 16 shows histological analysis of raphe formation in female VIC-Tgƒb1^{TG} mice.

FIG. 17 shows a Zipper model of right and left AV leaflets fusion and subsequent AV calcification in the pathogenesis of calcific aortic valve stenosis.

FIG. 18 shows increased elastin fragmentation in advanced nodular calcification of the aortic valve cusp region during calcific AS progression.

FIG. 19 shows fibrocalcific disease in stenotic AV valves of VIC-specific Tgƒb1 transgenic mice.

FIG. 20 shows increased osteochondrogenic proteins are associated with CAVD in the VIC-specific Tgƒb1 transgenic mice.

FIG. 21 shows increased TGFβ1 hyperactivation in cushion mesenchymal cells inhibits their apoptosis during heart development.

FIG. 22 shows increased TGFβ1 hyperactivation in valve interstitial cells leads to increased activation of SMAD-dependent signaling but results in dysregulated activation of components of non-canonical MAPK-dependent TGFβ signaling pathways.

FIG. 23 shows Western blot quantification of endogenously and transgenically produced TGFβ1 mature peptide, TGFβ1 unprocessed pro-peptide and total TGFβ1 in control and Tgƒb1^{TG};PostnCre (15-months-old) male and female mice.

FIG. 24 shows a table of antibodies.

FIG. 25 shows a table of qPCR Primers.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

The figures herein are for illustrative purposes only and are not necessarily drawn to scale.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press. Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March. Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The current disclosure generated valve interstitial cell-specific Tgƒb1 transgenic (Tgƒb1^{Tg}/PeriostinCre) mice by genetically intercrossing Tgƒb1^{Tg} mice (commercially available from the JAX Lab) and Periostin-Cre mice (obtained from Dr. Simon Conway. Indiana University School of Medicine as part of a collaboration, no MTA was filed).

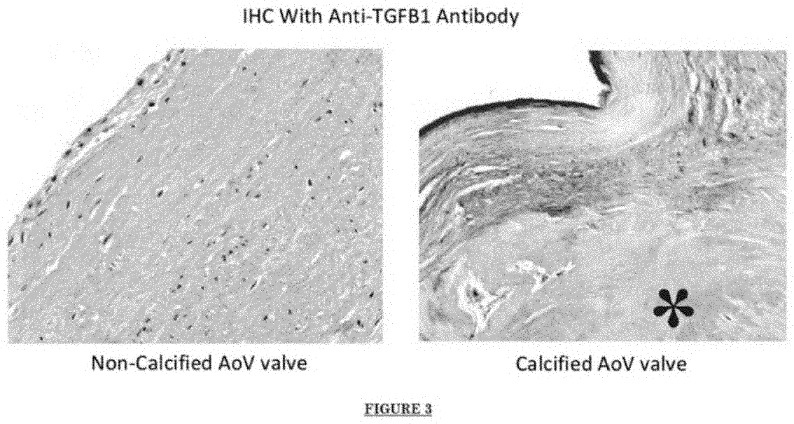

The results showed that although increased levels of bioactive TGFβ1 spontaneously causes CAVD in both genders, calcific AS is predominantly seen only in the male gender. Although male Tgƒb1^{Tg}/PeriostinCre mice were born with three aortic valve leaflets, over the year, the fusion of left and right coronary cusps of the aortic valve leaflets with an incomplete raphe underlie the pathogenesis of calcific AS. FIG. 3 shows transforming growth factor beta1 (TGFB1) is increased in human calcific aortic stenosis.

This establishes a novel paradigm that the functional bicuspid aortic valve with predominantly L/R coronary cusps fusion, which is often seen by clinicians in the surgical specimens of individuals with calcific AS, can be acquired during the pathogenesis of CAVD and necessarily may not be a congenital malformation as often anticipated in the current clinical practice. The data indicated that hyperactivated TGFβ1 signaling acting cell autonomously augmented cardiac neural crest cells colonization in the aortic valve region during embryonic heart development, which abnormally differentiated into an ectopic cartilage and laid the foundation of the CAVD in adults.

For the first time, the current disclosure identified that the calcific AS caused by TGFβ1 hyperactivation predominantly in the males was mediated by increased activation of SMAD2, SMAD3, SMAD1/5/9 and p38 MAPK; and decreased activation of pERK1/2 MAPK signaling pathways. Since increased bioactive TGFβ1 resulted in mildly calcified aortic valves but not calcific AS in the females, only SMAD1/5/9 activation was increased and that there was no significant changes in levels of activated SMAD2, SMAD3, p38 MAPK, and pERK1/2 MAPK. This indicated that TGFβ1 signaling via SMAD2, SMAD3, p38 MAPK, and pERK1/2 MAPK contributes to AS in the males whereas TGFβ1-induced activation of SMAD1/5/9 is involved in the valve calcification in both gender.

The current disclosure has also identified novel and unruly molecular and biochemical mechanisms that contributed to the pathogenesis of CAVD. Specifically, decreased NAU⁺/NADH-dependent polyADP-ribose polymerase (PARP1)—mediated ribosylation of SMAD3, and NAD+—dependent histone deacetylase (HDAC) Sirtuin 1 (SIRT1)—induced activation of osteoblast-specific transcription factor RUNX2 contributed to the CAVD caused by TGFβ1 hyperactivation.

The biochemical data also revealed that a novel potential interaction of transcription factors SMAD3, PARP1, SIRT1, and RUNX2 was involved in the pathogenesis of CAVD. Importantly, pharmacologic treatment by TGFβ receptor I kinase (SB431542) inhibitor significantly attenuated the initiation of CAVD in utera as well as the postnatal development of CAVD. TGfβ blocking antibodies, Tgƒb1-haploinsufficiency, and Smad3-haploinsufiiciency also significantly reduced the development of TGFβ1-induced CAVD in mice.

For the first time, these results successfully establish the causative role of TGFβ1 in development and pathogenesis of CAVD that recapitulates both age and gender-specific etiology associated with calcific AS in humans. For the first time, these results establish a reliable and convenient adult mouse model that ‘spontaneously’ develop calcific AS. These mice will be useful in developing and testing novel diagnosis, prevention and treatment strategies for the CAVD. Currently, there are no mouse models, which could effectively recapitulate age and gender specific development and progression of calcific AS. Our results have also uncovered novel developmental, cellular, molecular, biochemical pathways and approaches for therapeutic targeting to block the development and/progression of CAVD in mice. Novel observations are likely to emerge from these findings which will favorably shift the current paradigm and approaches for an early detection and prevention and strategies for targeting specific pathways for a potential medical treatment of CAVD as well as calcific AS progression in older humans.

Overall, these results successfully establish the causative role of TGFβ1 and underlying pathogenic mechanisms involved in the development and pathogenesis of CAVD that recapitulates both age and gender-specific etiology associated with calcific AS in humans.

This model will be used to test novel small molecule inhibitor compounds, antibodies, ligand-traps which are either commercially available or obtained from industry (via MTA). Successful application will result in further testing of the select compounds in larger animal model and/or in human safety trials.

Our results established a reliable, convenient, age-appropriate, adult mouse model that ‘spontaneously’ develop calcific aortic valve disease and recapitulate gender-specific progression and L/R coronary leaflet fusion resulting in calcific aortic stenosis. No experimental insults such as hypercholesterolemia, hypertension, diabetes, and dietary supplements (vitamin D, high phosphate, vitamin K) are required to induce CAVD or aortic stenosis in vivo in Tgƒb1^{Tg} model. It is anticipated that our novel mouse model will have significant implications in translational research for developing effective strategies of an early detection and prevention of CAVD, and targeting molecular pathways for a potential medical treatment of ‘symptomatic’ calcific aortic stenosis in older humans.

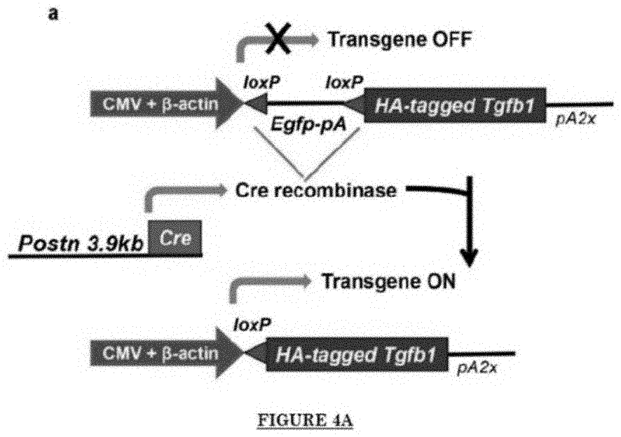

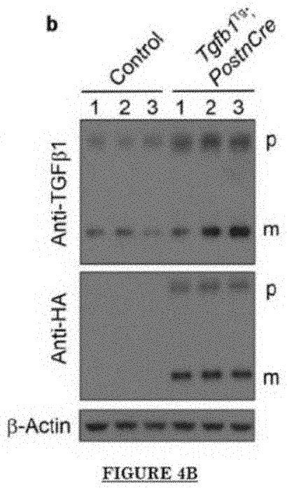

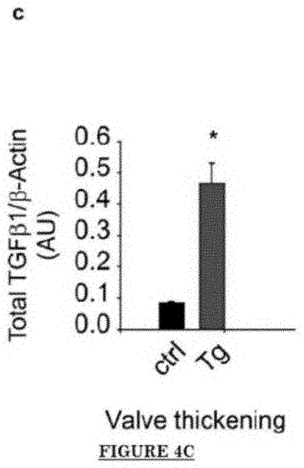

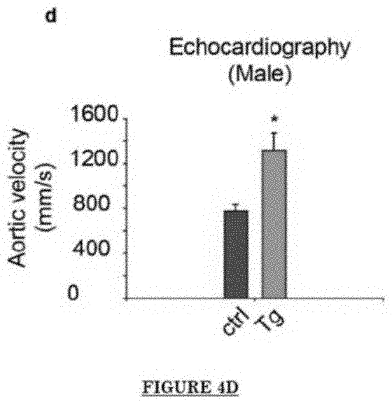

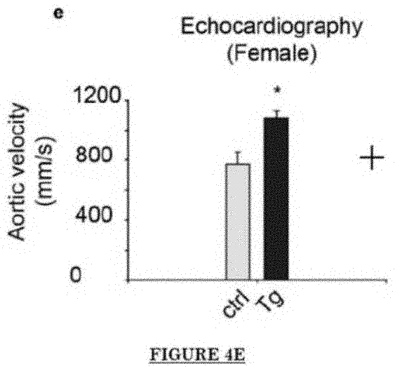

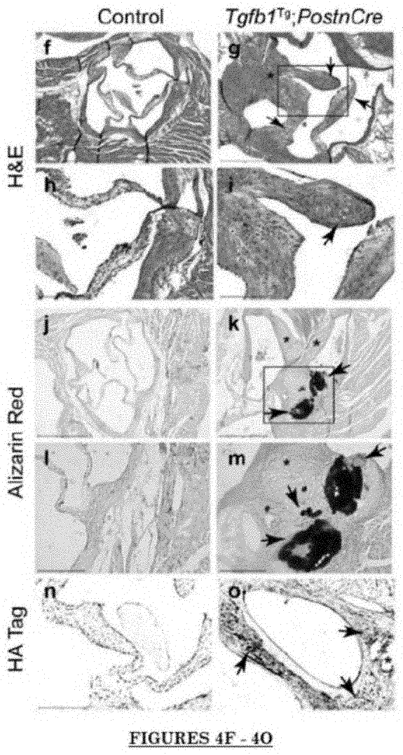

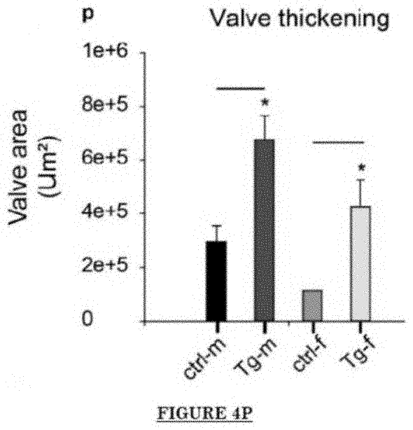

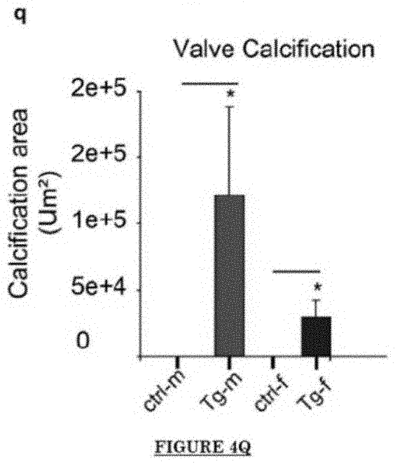

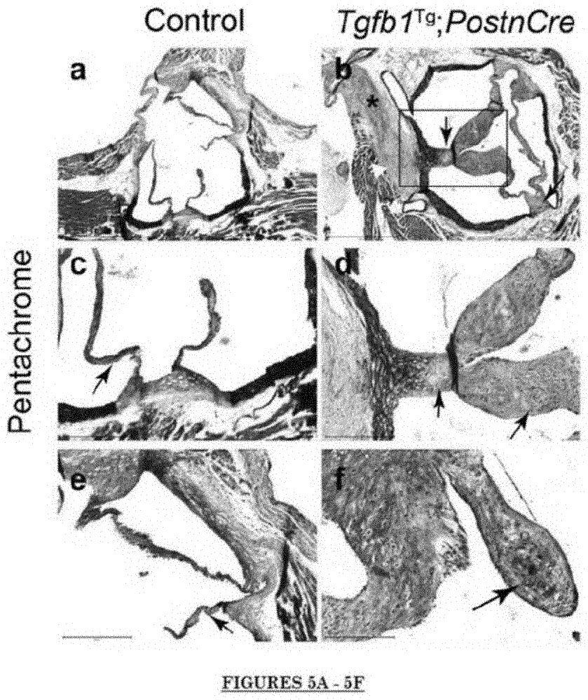

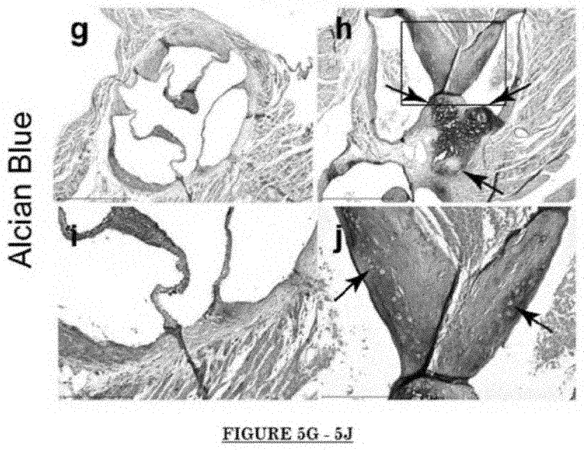

Valve interstitial cell-specific Tgƒb1 conditional transgenic (Tgƒb1^{TG};PostnCre) mice were produced by intercrossing the Tgƒb1 and Periostin Cre (PostnCre) mice (FIG. 4A). In the Tgƒb1 transgene, the Tgƒb1 cDNA (hemagglutinin (HA) tagged) was mutated (Cysteine 223, 225 to Serine) to prevent the assembly of the latent associated peptide (REF). This would allow direct binding of the secreted active dimeric TGFβ1 mature peptide to the TGFβ receptors (REF). Western blotting of dissected aortic valves using TGFβ1 and HA epitope tag antibodies confirmed the overexpression of the bioactive HA epitope-tagged TGFβ1 in both male and female Tgƒb1^{TG};PostnCre mice (FIGS. 4B-4C).

Immunohistochemical detection showed that VIC produced HA epitope-tagged-TGFβ1 in the aortic valves and the surrounding periaortic fibrous tissue of Tgƒb1^{TG};PostnCre mice (FIGS. 4D-4E).

Histological and morphometric comparison revealed significantly thickened aortic valves in 12-months old male Tgƒb1^{Tg};PoslnCre mice compared to the age and gender matched non-transgenic control mice (FIG. 4). Approximately 1/5 cases of female Tgƒb1^{Tg};PoslnCre mice at 12 months of age showed mild aortic valve thickening compared to the age and gender matched control mice. Echocardiography showed significantly increased peak aortic velocity in the Tgƒb1^{Tg};PostnCre mice. MicroCT analysis confirmed the presence of calcification in vivo in the aortic valve region of the heart (FIG. 4). These mice were born with normal tricuspid aortic valve morphology (FIG. 4). Importantly, right and left coronary leaflets of the aortic valve were predominantly fused at the base and formed a raphe (4/4 cases of male) resulting in a functional bicuspid aortic valve-like morphology (FIG. 4). The overall length of the raphe (from annulus to separation of the cusps) increased >2-fold (P<0.05) in Tgƒb1^{Tg};PostnCre mice which developed symptomatic CAS.

Alizarin red and pentachrome staining revealed significant calcific deposits and the presence of cartilage in the aortic valve of older Tgƒb1c^{Tg};PostnCre mice (FIG. 4). There was no BAV morphology, aortic valve calcification, and ectopic cartilage formation in age and gender matched control mice (FIG. 4). One distinguishing feature of CAS in Tgƒb1 ^{Tg};Postncre mice was the asymmetric initiation and progression of calcification during disease progression. Calcification began at the base of aortic valve, near the attachment to the aortic wall in aortic annulus and periaortic region, and extended towards to cusp region in older mice (FIG. 4). This AV calcification has significant resemblance to the aortic valve calcification reported in CAS patient 25. Histological and morphometric studies also revealed that all 12-mo-old female Tgƒb1^{Tg};PostnCre mice had mild to moderate calcification and thickening of AoV but there was no aortic stenosis (FIG. 4). Also, the extent of aortic valve calcification was significantly less in females compared to the male Tgƒb1c^{Tg};PostnCre mice (FIG. 4). Thus, the data indicate that the extent of aortic valve thickening and calcification was significantly higher in older transgenic males compared to the age-matched females Tgƒb1^{Tg};PostnCre mice (FIG. 4). Our data also indicate that female mice are protected against TGFβ1-induced CAS. These findings are striking similar to the reported clinical findings in human population affected with CAVD.

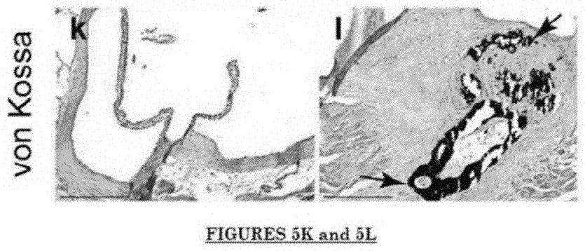

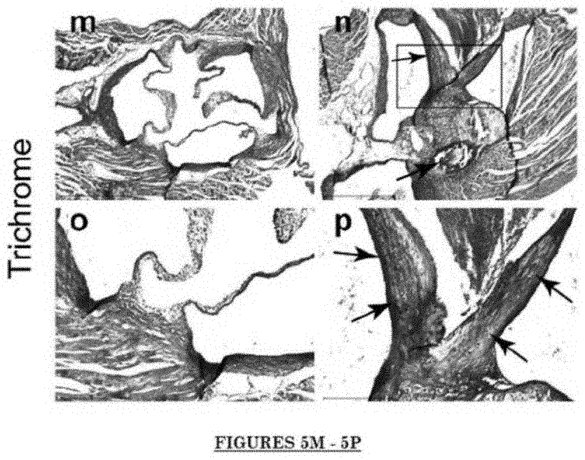

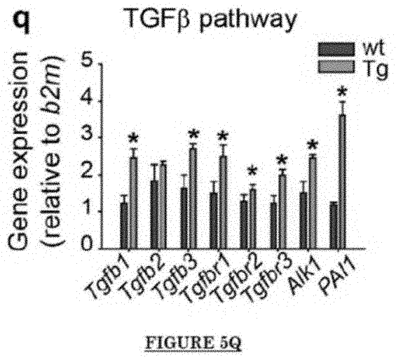

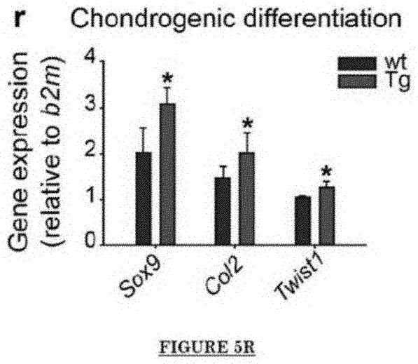

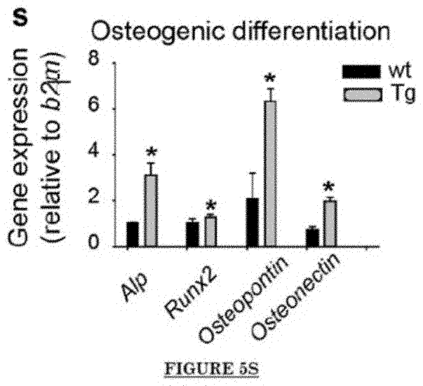

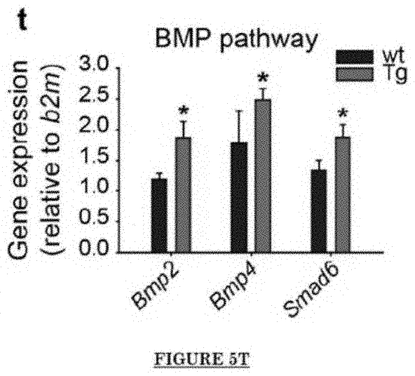

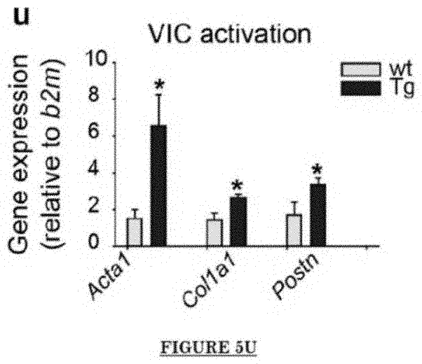

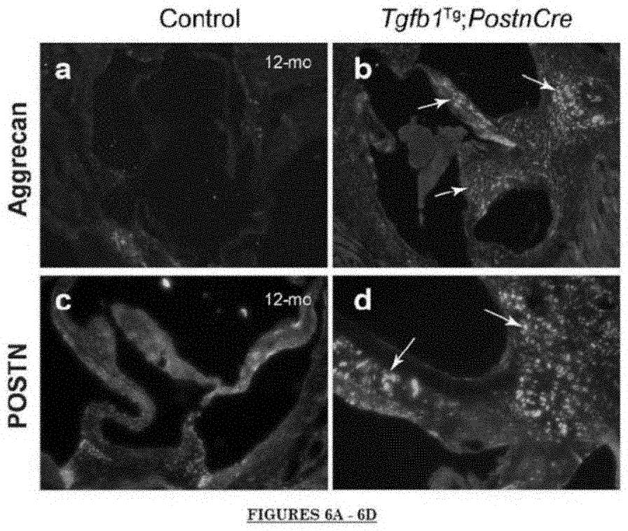

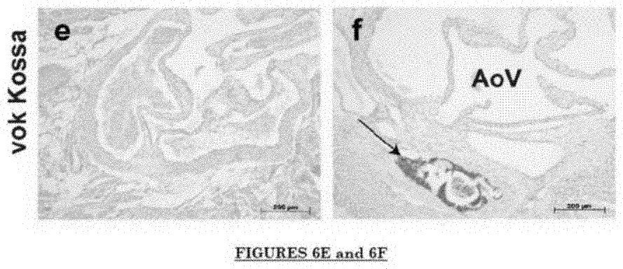

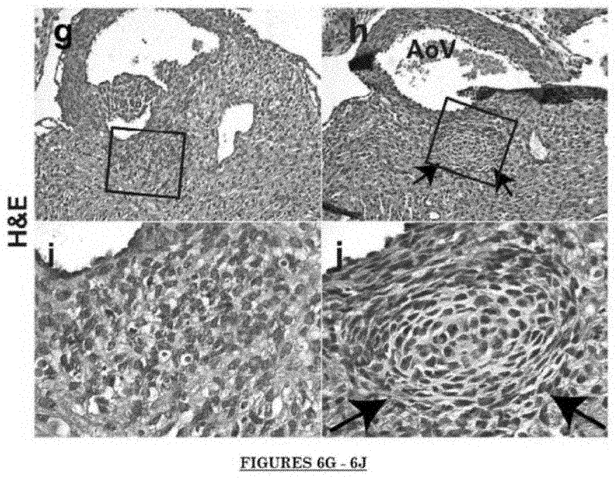

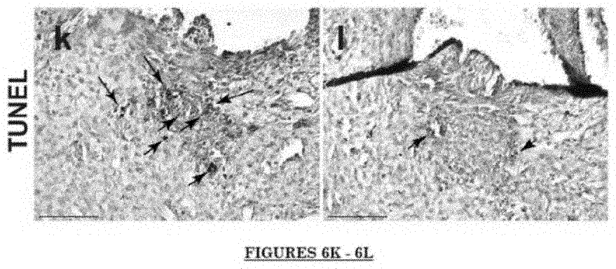

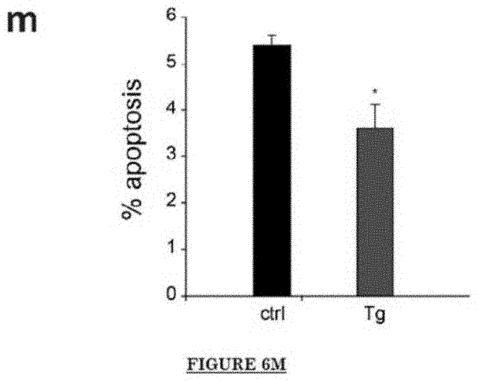

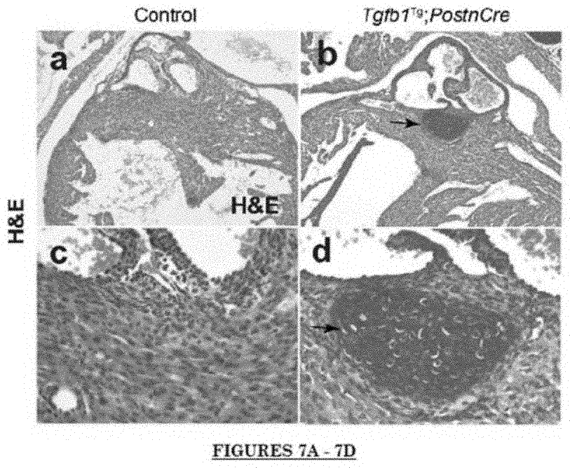

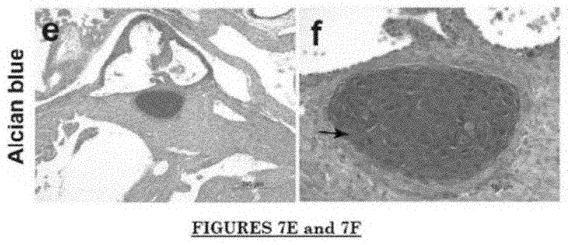

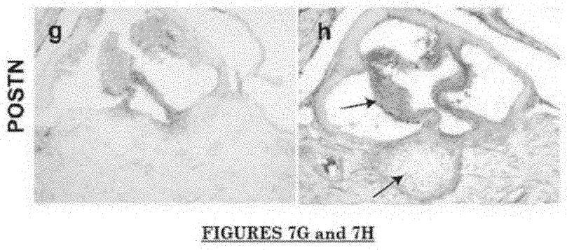

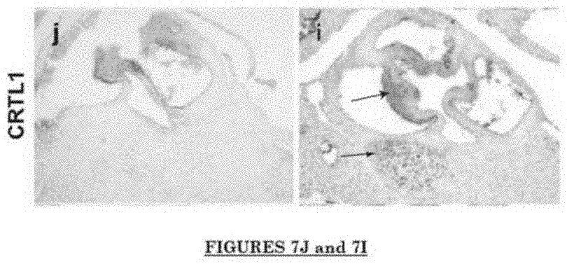

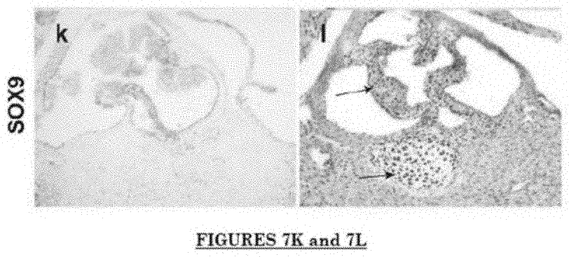

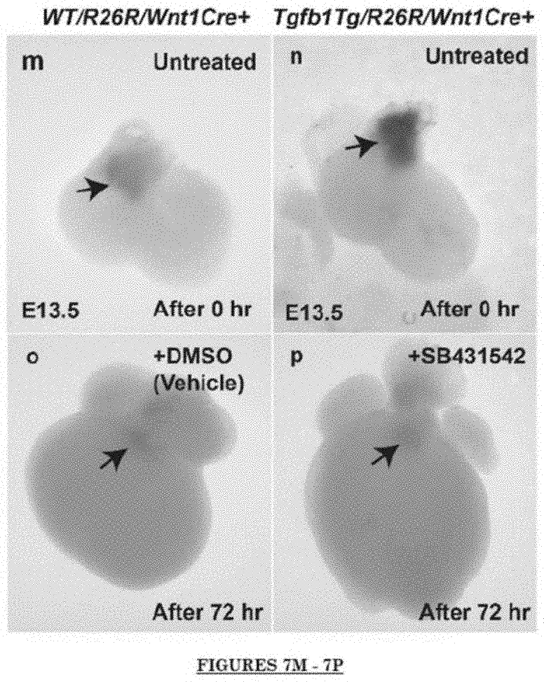

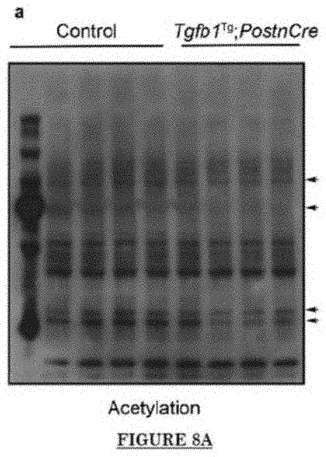

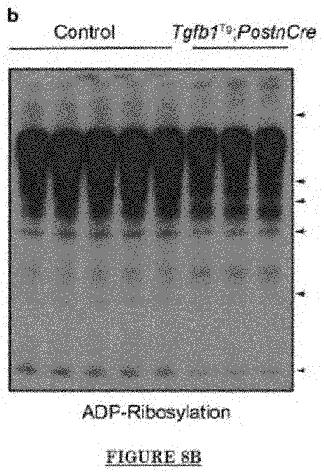

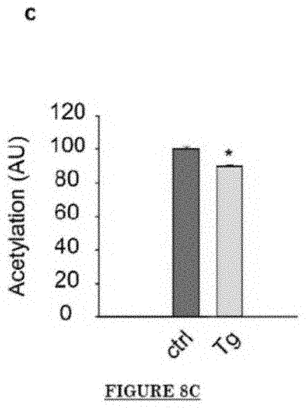

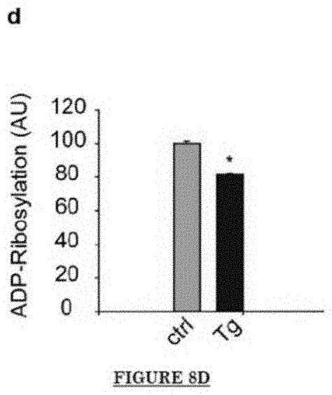

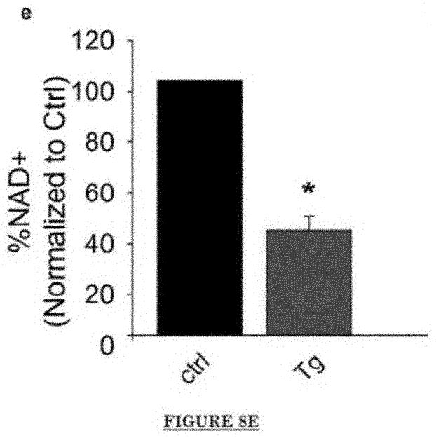

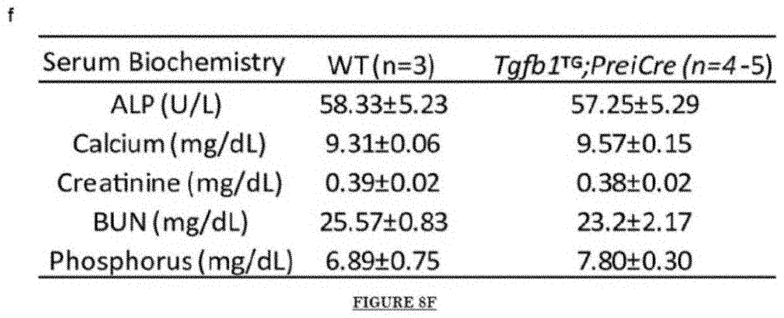

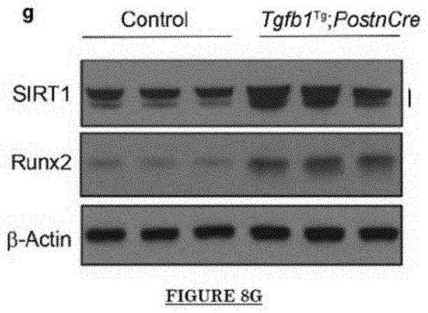

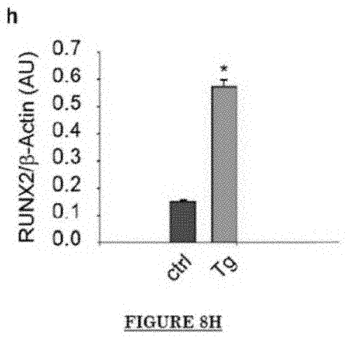

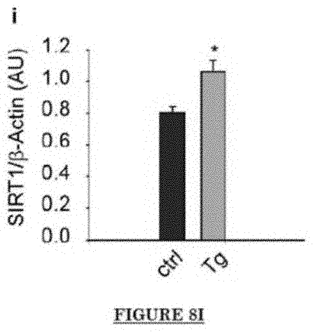

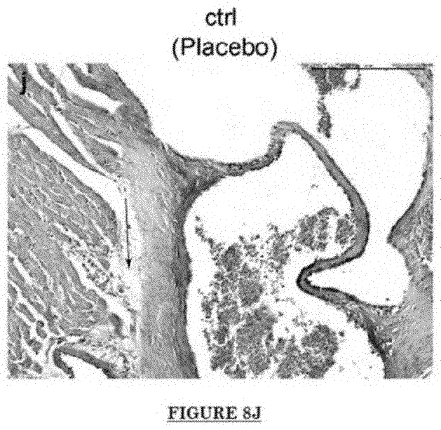

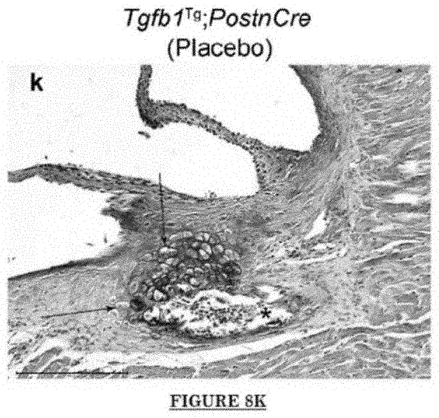

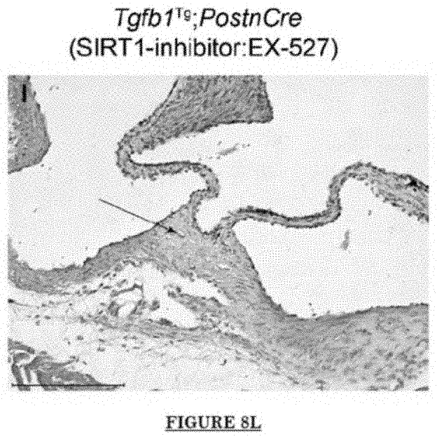

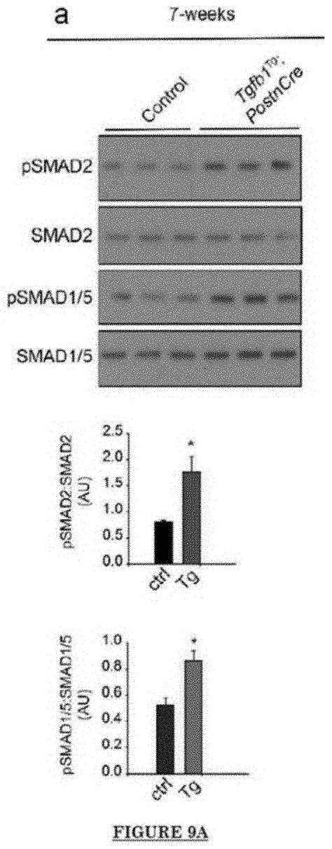

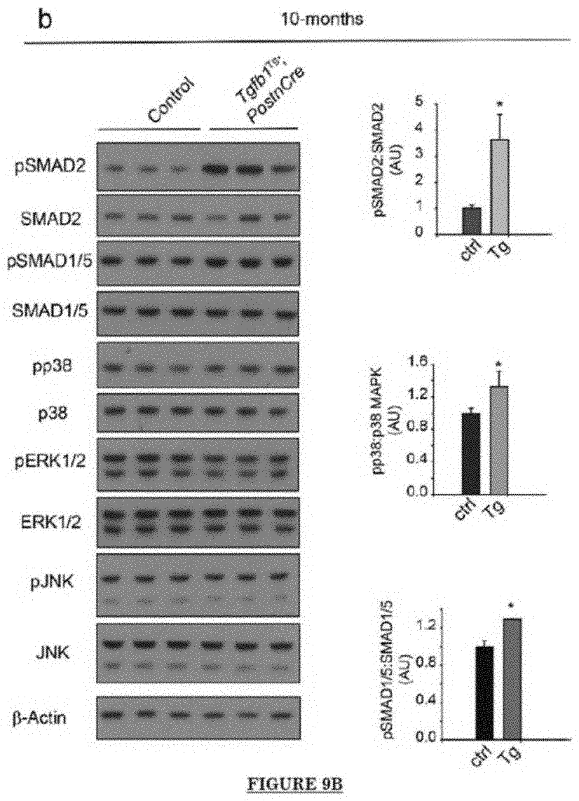

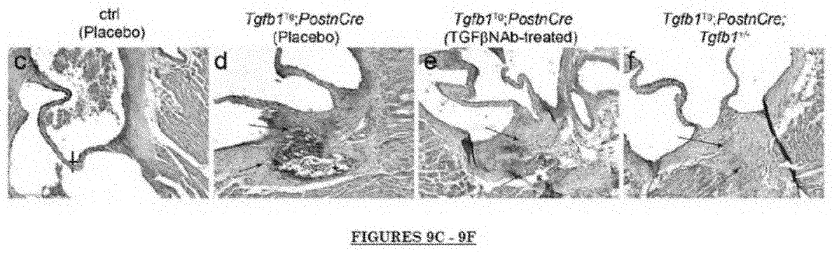

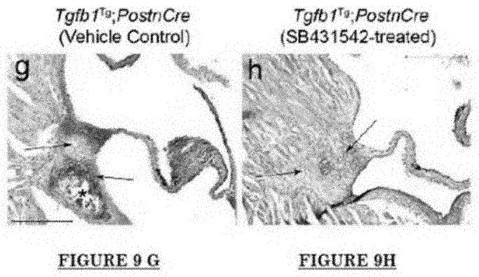

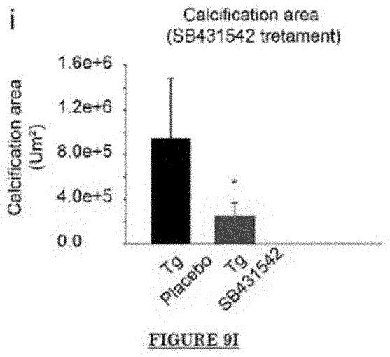

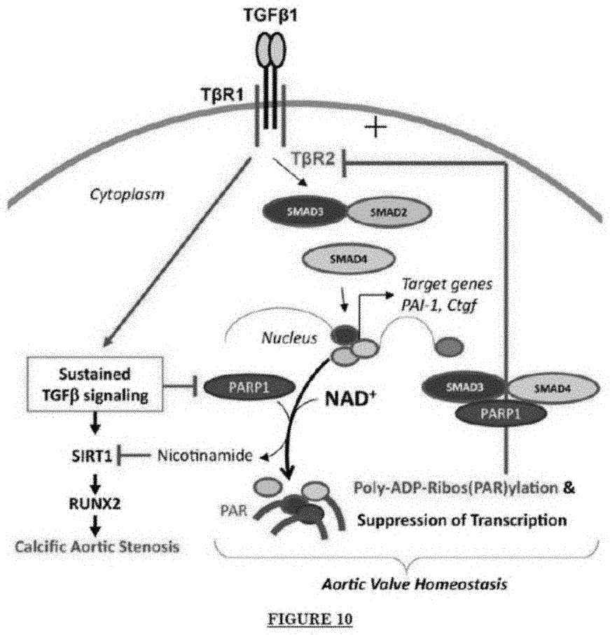

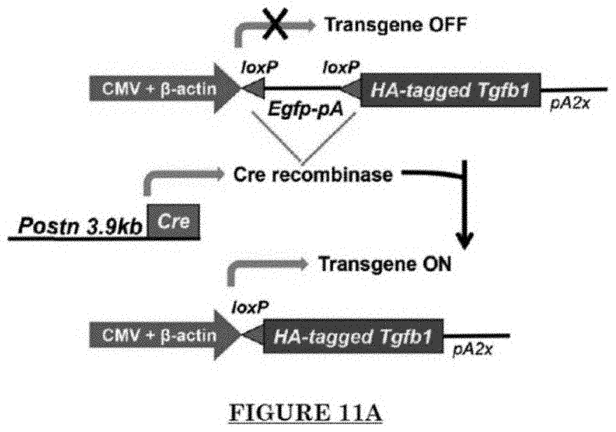

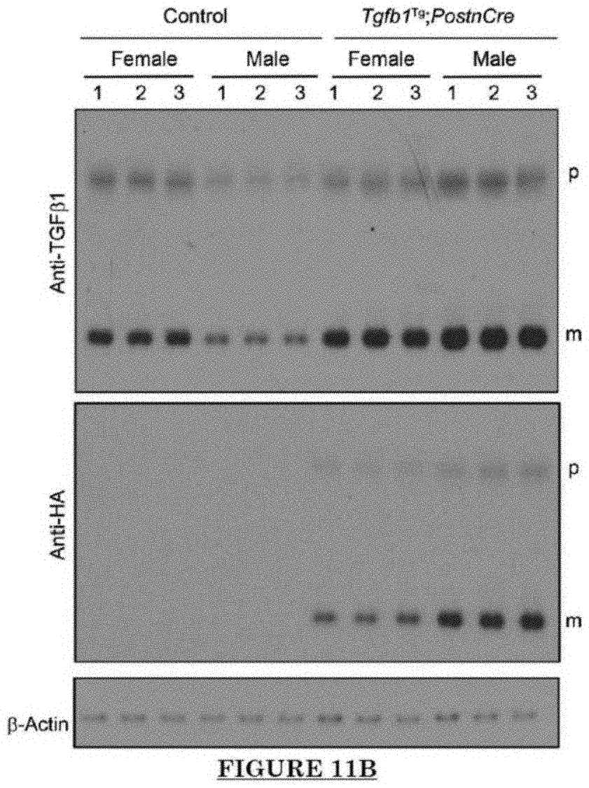

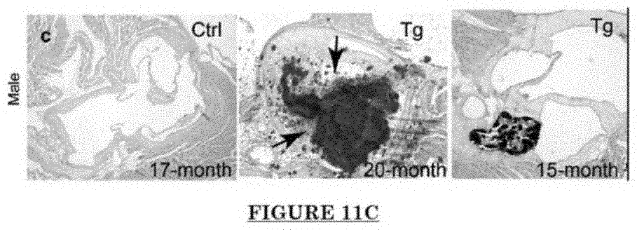

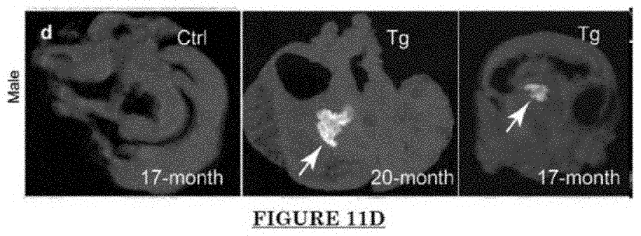

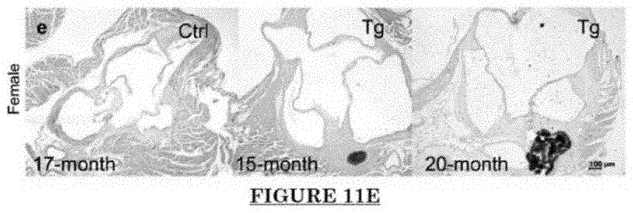

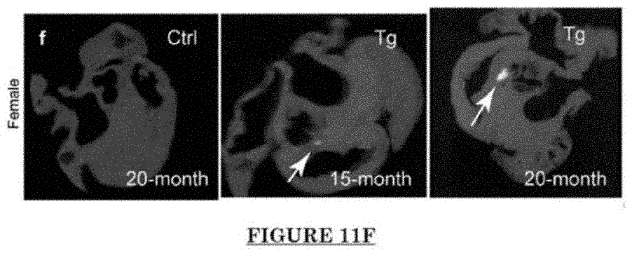

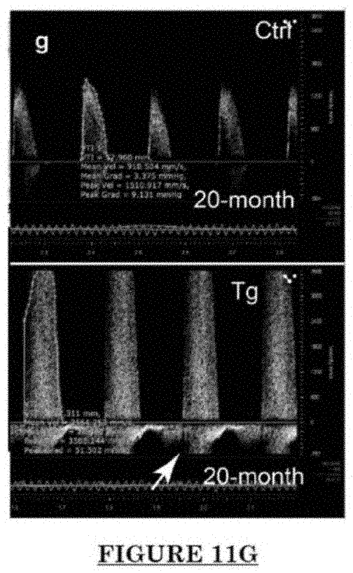

Microcomputed tomography (microCT) confirmed the significant presence of AV calcification in vivo in adult male Tgƒb1^{Tg};PostnCre mice (FIG. 5) Aortic valve hemodynamics, as assessed by echocardiography in a longitudinal study, (representative images of peak aortic jet velocity for Tgƒb1^{Tg} mice, FIG. 5, bottom tracings) was consistent with the presence of AS in 12-mo-old Tgƒb1^{Tg};PostnCre mice. The peak aortic jet velocity, the peak transvalvular pressure gradient, and the mean transvalvular pressure gradient were all markedly increased in Tgƒb1^{Tg};PostnCre mice versus age and gender matched control mice. The data showed that baseline cardiac parameters (ejection fraction and fractional shortening) were not affected in adult Tgƒb1^{Tg};PostnCre mice with asymptomatic CAVD but significantly reduced in the older male Tgƒb1^{TG};PostnCre mice which developed symptomatic calcific AS (FIG. 5). Aortic valve sclerosis and calcification was noted as early as 4-weeks of age in Tgƒb1c^{Tg};PostnCre mice (FIG. 5). Calcification started to spread deeper into the basal region of AV leaflet and progressed with the age in Tgƒb1c^{Tg};PostnCre mice (FIGS. 5C-5F; FIG. 6B, 6F, 6D, 6H). Echocardiographic analysis in a longitudinal study followed by histological and morphometric analyses confirmed that aortic valve stenosis, fibrosis, and calcification increased with age in male Tgƒb1^{Tg};PostnCre mice.

Confocal laser scanning multi-photon microscopy via second harmonic generation and picosirius red histochemistry were used for visualizing and relative quantification of collagen fibers. In control mice, collagen at the base of the cusps was oriented primarily parallel or perpendicular to the long axis of the attachment site. In Tgƒb1^{Tg};PostnCre mice, there was a mesh of collagen at the base of the valve cusps, oriented around a 45° angle to the attachment of the cusps, which tethered adjacent cusps to one another. Thus, although total collagen in the valve was increased in Tgƒb1^{Tg};PostnCre mice, collagen was remodeled at the base of the valve in Tgƒb1^{Tg};PostnCre mice to form a mesh that spans adjacent cusps, which may restrict opening of the valve resulting symptomatic aortic stenosis in males. In addition, both picosirius red and trichrome staining indicated significant age-dependent increase in fibrosis in aortic valves, aortic root, and periaortic region in Tgƒb1^{Tg};PostnCre (FIG. 5).

To determine the developmental and cellular origin of the CAVD in adult mice, Tgƒb1^{Tg};PostnCre mice were analyzed during heart development and postnatal stages (neonates, juvenile, adult). The data showed that PostnCre-derived endocardial cushion cells producing bioactive TGFβ1, acting cell autonomously, were trans-differentiated into mesenchymal cell aggregate or nodule at the periaortic region. The condensed outer mesenchyme served as the perichondrium, gradually formed and moved inward as chondroblasts/chondrocytes, which ultimately differentiated into mature chondrocytes. The hypertrophied chondrocytes eventually calcified around 3 4 weeks of age. TUNEL analysis indicated that Tgƒb1-overexpressing endocardial cushion cells escaped normal apoptosis, which occurs during outflow tract remodeling and AV development. Cell proliferation studies indicated sustained cell proliferation in TGFβ1-overexpressing VIC in the postnatal heart.

We also determined the impact of increased bioactive TGFβ1 on the fate of cardiac neural crest (NC) cell lineage (via Wnt1Cre). Wnt1Cre+mice is very well characterized Cre driver for migratory and post-migratory neural crest cells. Importantly, cell lineage mapping analysis showed that NC contributed significantly to peri-aortic and AoV-hinge region of the four-chambered fetal heart. Histological analysis and cell lineage mapping revealed the enhanced NC colonization in the outflow tract of Tgƒb1 ^{Tg}; Wnt1Cre+mice (n=3), which resulted in ectopic intra-cardiac cartilage nodule formation in the periaortic region of the fetal hearts. By using embryonic heart cultures and NC lineage tracing analysis (via R26RlacZallele) the data showed that the enhanced NC colonization in Tgƒb1^{Tg};Wnt1cre+embryos was rescued compared to control embryos by blocking TGFβ receptor-dependent signaling (via TGFβR1 kinase inhibitor, SB431542). Tgƒb1^{Tg};Wnt1Cre+mice died at perinatal stage due to severe craniofacial defects, which precluded the analysis of development and progression of CAS in the adult mice. Collectively, the data indicate that increased TGFβ1 activation in endocardial cushion or valve progenitor cells of neural crest origin triggers a pathological onset of AV disease during embryonic heart development, which manifests as CAVD in adult mice.

There is evidence that TGFβ1 promotes SMAD3 and poly-ADP(ribose) poly-merase (PARP1) interaction to target gene promoter for transcriptional repression and TGFβ receptors are important targets of PARP1-dependent transcriptional repression and post-translational modification, which serve as important mediator of feedback mechanism. Because cellular levels of NAD⁺ and [NAD]⁺/[NADH] dictate PARP1-dependent signaling events through NAD-dependent histone deacetylase (HDAC) enzyme sirtuin 1 (SIRT1) activation, we measured cellular NAD+/NADH levels and PARP1-dependent posttranslational modifications. PARP1 activation drives poly-ADP-ribosylation along with concomitant increase in total cellular acetylation. Western blot analysis of AV tissue revealed that ADP-ribosylation and protein acetylation was significantly decreased in the adult (8-month old) Tgƒb1 ^{Tg};PostnCre mice compared to control animals. Importantly, Tgƒbr2, which is authentic transcriptional target of SMAD3-PARP1 repressor complex, was significantly increased in the AV tissue of adult Tgƒb1^{Tg};Postn(re mice, indicating that sustained inhibition of PARP1 de-repressed TGFβ signaling. Since PARP1 can inhibit SIRT1 (REF), our data also revealed significantly increased SIRT1 and RUNX2 in AV tissue of adult Tgƒb1^{Tg};PostnCre mice. RUNX2 is master regulator of calcific aortic valve disease and regulated by SIRT1. In vivo pharmacologic inhibition of SIRT1 activity by EX 527, a potent and selective small-molecule inhibitor of SIRT1 catalytic activity, significantly rescued the development of calcific AV disease in Tgƒb1^{Tg};PostnCre mice. Thus, these results indicate that sustained TGFβ1 hyperactivation leads to PARP1 inhibition resulting in enhanced SIRT1, which contributes to higher RUNX2 and calcification in the AV.

To determine if SMAD3 physically interact with PARP1, SIRT1, and RUNX2, coimmunoprecipitation analysis was done which confirmed the presence of these molecules in a shared transcription factor complex. Downstream canonical and non-canonical molecular signaling mechanisms of TGFβ1 hyper-activation was also determined by western blot analysis. The data identified significantly increased activation of SMAD2, SMAD3, and SMAD1.15/8, indicating hyper-activated canonical TGFβ signaling mechanisms in the AV tissue of adult male Tgƒb1^{Tg};Postn(re mice. In females, there was no significant changes in SMAD2/3 activation but SMAD1/5/9 was activated.

Consistently, TGFβ target gene expression (Pail, Col1a1(Smad7) were also significantly increased in the AV of the adult Tgƒb1^{Tg};PostnCre mice. Extensive molecular analysis of non-canonical TGFβ signaling mechanisms indicated that MAP3K7 (i.e., TAK1) and p38 MAPK but not the SAPK/JNK were significantly activated in the AV of male Tgƒb1^{Tg};PostnCre mice. Importantly, levels of activated p44/42 MAPK (ERK1/2) in the AV were significantly downregulated in the Tgƒb1^{Tg};PostnCre mice.

Next, we showed that the Tgƒb1^{Tg} mice have only cartilage or chondrogenic and fibrous lesion at 3 weeks but they develop fibrocalcific lesions in AV region at 4 weeks of age. Thus, we intraperitoneally (i.p.) injected control and Tgƒb1^{Tg} mice with TGFβR1 or ALK5 kinase inhibitor (SB431542) starting at 3 weeks and analyzed them at 7 weeks of age for development of fibrocalcific lesions. The data revealed that in vivo pharmacologic inhibition of TGFβ signaling by SB431542 treatment attenuated the chondrogenic and fibrocalcific lesion formation in the AV region of the Tgƒb1 Tg mice. Furthermore, superimposed heterozygous deletion of Tgƒb1 in adult Tgƒb1^{Tg};PostnCre mice rescued the development and progression of chondrogenic and fibrocalcific lesions in the AV of Tgƒb1^{Tg};PostnCre;Tgƒb1+/−compound mice compared to Tgƒb1^{Tg};PostnCre mice. Further, Smad3 heterozygous deletion also attenuated the development of CAVD. Collectively, these results indicate that selective therapeutic partial reduction of TGFβ1 or TGFβ signaling is a novel strategy to attenuate development and progression of CAVD in vivo.

We used conditional transgenic mice to investigate the relationship between CAS and TGFβ1. Our major findings are the following: (1) valve-interstitial cell-specific overexpression of the constitutively activated form of TGFβ1 causes CAVD in both gender but only males developed CAS in adult mice; (2) VIC Tgƒb1^{Tg} mice are born with tricuspid AV but acquire BAV morphology by RC and LC fusion; (3) activated TGFβ1 helps the outflow tract endocardial cushion cells to escape the apoptosis, resulting in abnormal outflow tract cushion remodeling and extopic intra-cardiac cartilage formation during heart development; (4) intra-cardiac cartilage undergoes cartilage hypertrophy and calcification at 4 weeks after birth; (5) endocardial cushion cells of neural crest origin is identified as the major cell type responsible for chondrogenic differentiation in response to cell autonomous action of the activated TGFβ1; (6) TGFβ1 hyper-activation causes age-dependent fibrocalcific changes in the AV resulting in the progression from AVS to CAS in males; (7) bioactive TGFβ1 overexpression leads to increased activation of SMAD1/518 in both gender before AV calcification but exhibit increased activation of SMAD2/3 associated with fibrocalcific changes in pathogenesis of AS only in the adult males, indicating that TGFβ1-dependent activation of SMAD1/5/8 is contributes to for AV calcification in both gender whereas SMAD2/3 activation contributes to AS in males; (8) under sustained TGFβ signaling, inhibition of PARP1 and cellular NAD⁺ allows SIRT1 activation resulting in enhanced RUNX2 and calcification or osteogenesis; (9) pharmacologic inhibition of TGFβ signaling or SIRT1 activation attenuate development of CAVD; (10) genetic heterozygous deletion of Tgƒb1 in VIC Tgƒb1^{Tg}; mice significantly rescued the development of CAVD. Our results support a causal relationship between increased TGFβ1 and development and progression of CAVD. This is consistent with clinical evidence that patients with calcific AS have increased TGFβ1 (REF). This is the first study that established the in vivo role of TGFβ1 in development and pathogenesis of calcific AS. Until now, the etiology of CAVS has remained entirely obscure, and there has been no convenient animal model, which survive to an old age, in which to study CAVS pathology without any additional experimental insults/injury including hyperlipidemia, hypercholestrolemia, hypertension, diabetes, chronic kidney disease, vascular/arterial calcification, vitamin-k-treatment, vitamin-D-diet.

Calcific AS is the consequence of progressive fibro-calcific remodeling on initially normal (tricuspid) AV or a congenitally abnormal (BAN) AV. The BAV accounts for nearly half of the AVs that are surgically removed due to calcific AS. In humans, the fusion of the RC and LC AV leaflets leads to BAV formation (REF). However, in most mouse models of BAV, the NC AV leaflet is normally fused with RC or LC leaflets. The present study, which found evidence of fusion between RC and LC leaflets of the AV with elevated VIC TGFβ1 in adult mice, fits in well in suggesting that VIC Tgƒb1^{Tg} mice is the first transgenic mouse model that closely resemble human BAV pathology associated with calcific AS.

Pathogenesis of BAV is unclear. Multiple factors are involved in AV development. BAV has developmental and genetic basis. Animal studies suggest the involvement of NC in the development of AV. The fusion of the right and left coronary cusps (type 1 BAV) is found in β0% cases of BAV. Age and gender are significant factors leading to fibrocalcific AS. Men are at a higher risk of CAS than women (REF). This study establish that TGFβ1 plays an important role in gender disparity associated with the pathogenesis of CAVD. Importantly, specific components of TGFβ signaling pathways are altered in male vs female in response to TGFβ hyperactivation which drives LC and RC fusion resulting in AS.

For a long time, calcific AS was thought to be a ‘degenerative’ process caused by time-dependent wear-and-tear of the leaflets and passive calcium deposition. Our results provide direct and compelling evidence that calcific AV disease also has strong developmental component and identified increased TGFβ1 in VIC of predominantly NC origin as an important contributor to the development and progression of calcific AS. Pathological conditions such as hyperlipidemia, hypercholestrolemia, hypertension, diabetes, chronic kidney disease, and vascular/arterial calcification strongly increase the risk of developing CAVS (REFS); however, none of these changes appears to be required for development of the CAS, underlining its multifactorial pathogenesis. Notably, increased TGFβ1 has been implicated in many of these pathologies, and therefore can serve as a mediator of CAVS under these pathological conditions. Indirect evidence has been accumulating that inflammatory mediators may be important in the progression of CAVS and other cardiovascular diseases (REF). Fibrosis is also a strong risk factor for end-stage CAVS and can result in deposition of calcium minerals and the formation of fibrocalcific lesions at a young age (REF). Our data indicate that TGFβ1 is a key cytokine involved in the fibrocalcific aortic stenosis. Studies of the Klotho KO model(REH) indicate that, at least in mice, aging is associated with increased activity of the BMP pathway (i.e., SMAD1/5/9) and that increased activation of BMP signaling (via SMAD1/5/9) alone is not sufficient to induce CAVS (REF).

These findings strongly suggest additional signaling inputs that can activate and/or synergize with SMAD1/519 are critical for the development of CAVS. We have shown that chronic endocardial cushion cell overexpression of bioactive TGFβ1 can result in the activation of both SMAD2 and SMAD1/519, increased TGFβ/BMP signaling, and calcific deposits in vivo, indicating that hyperactivated TGFβ signaling acting in autocrine fashion in endocardial cushion mesenchymal cell lineage is important in both development and progression of CAVS. Finally, TGFβ1 signaling may mediate the CAVS indirectly because of its capacity to interact with anti-aging molecules such as Klotho (REF), which protects against CAVS by restraining pathological activation of TGFβ signaling (REF). Although Klotho KO mice also spontaneously develop AV calcification at 2-months of age, they do not survive beyond 2 months of age that limit their potential use in further studies.

Although SIRT1/NAD⁺-dependent activation drives calcification via RUNX2 (REF), our data also suggest that inhibition of PARP1 and not the cellular modulation of NAD⁺ can regulate SIRT1 activity which is responsible for the AV calcification. This suggestion is consistent with the finding that despite initial consumption of NAD⁺, PARP1 activation triggers the induction of NAD⁺-regenerating enzymes to replete cellular NAD⁺ while keeping SIRT1 inhibited through sustained generation of nicotinamide. Further, PARP1 senses and responds to oxidative, and environmental stresses by hydrolyzing NAD⁺ to nicotinamide and ADP-ribose resulting in the activation of cellular repair and protective pathways. Based on our data, under sustained TGFβ signaling, inhibition of PARP1 depletes cellular NAD⁺ due to the inhibition of regeneration of NAD⁺. Thus, our results raise the possibility that PARP1 activation via induction of protective stress response through inhibition of SIRT1 along with the upregulation of cellular NAD⁺ will be a beneficial therapeutic strategy for. CAS.

This study presents the Tgƒb1^{Tg} mouse as a novel and unique preclinical model of CAVS. This model clearly indicates developmental origin of CAVS and identify endocardial cushion cells of NC lineage as the critical cell type involved in the onset and development of CAVS. This model does not require any additional experimental insults (i.e. hyperlipidemia, hypercholesterolemia, hypertension, diabetes) for inducing GAS. Several characteristics of this model reiterate the pathogenic and gender-specific features of CAVS in humans and thus may help understand the pathophysiological and molecular mechanisms underlying the development of CAVS. TGFβ1 signaling intervention to identify druggable targets in this unique and novel animal model of age-dependent and gender-specific CAVD progression will be useful for the evaluation of novel pharmacotherapies for calcific AS prevention or treatment.

Generation of the mice model: All animal works were performed in accordance to approved protocol by Institutional Animal Care and Use Committee (IACUC) at University of South Carolina. Tgƒb1^{Tg},+; Peri CreTg,+mice and littermate controls were generated by mating Tgƒb1^{Tg} female mice (REF) with Peri CreTg,+male mice (REF). Tgƒb1^{Tg},+; Peri CreTg,+;Tgƒb1+/−mice were generated by mating Tgƒb1+/−female mice to Tgƒb1 ^{Tg},+; Peri CreTg,+male. In the transgenic mice, Tgƒb1 cDNA contained a fully bioactive hemagglutinin (HA) epitope tag which distinguished the transgenic bioactive TGFβ1 from the endogenous TGFβ1. The Tgƒb1 transgene expression requires Cre-mediated deletion of an intervening foxed enhanced green fluorescent protein (EGFP) gene in order for a ubiquitous β-actin promoter to transcribe constitutively active HA tagged Tgƒb1 cDNA. PostnCre mice express the Cre recombinase predominantly in the cardiac outflow tract endocardial cushion cells (E10.0) and adult VIC.

Genotyping was performed using standard protocol for DNA extraction and purification of tail tissue followed by polymerase chain reaction to identify Tgƒb1 and Cre transgenes using primer( ) and primer respectively.

Western blotting was performed with heart and aorta samples from different control and experimental mice. The tissue samples were weighed, cut into small pieces, and homogenized using Wheaton tapered tissue grinders (Thermo Scientific, Rockford, Ill., USA) in M-PER mammalian protein extraction reagent (Thermo Scientific) with complete mini protease inhibitor cocktail (Sigma). Following homogenization lysate was subjected to brief sonication for 20 sec in ice and kept at room temperature for 20 mins. Then centrifugation was performed at 15,000 rpm for 20 min at 4° C. and the supernatants were collected. Total protein concentration in the supernatant was determined using Pierce BCA protein assay kit (Thermo Scientific. Rockford, Ill., USA) and the samples were stored at −80° C. until further use. Western blotting was performed, as described previously, using these protein samples and the primary IgG antibodies against pSMAD2, SMAD2, pSMAD1/5, SMAD1, pp38, p38, pTAK1, TAXI, pJNK, JNK, pERK1/2, ERK1/2 etc. at a dilution of 1:1000. Primary IgG antibodies against all these proteins were procured from Cell Signaling (Cell Signaling Technology, Inc., Danvers, Mass., USA). The horseradish peroxidase conjugated anti-mouse or anti-rabbit secondary IgG antibody (Cell Signaling) was used at 1:5000 dilution to detect a primary IgG antibody. Western blots were incubated with Clarity western ECL detection reagents (Bio-Rad laboratories) and exposed to X-OMAT AR films (Eastman Kodak, Rochester, N.Y., USA) for autoradiography. The autoradiograms were scanned on an EPSON Scanner using Photoshop software (Adobe Systems, Seattle, Wash., USA). β-actin, clone AC-15 monoclonal primary anti-body (Sigma-Aldrich, St. Louis, Mo., USA) was used as loading control to compare equal loading in the SDS-PAGE.

NAD+/NADH quantification

B1Tg/Peri-Cre mice heart samples (˜20 mg) were washed in cold PBS and homogenized in 400 μl of NADH/NAD extraction Buffer (BioVision, Milpitas, Calif., USA) in a Wheaton tapered tissue grinders (Thermo Scientific, Rockford. IL, USA). Then tissue homogenates were centrifuged at 14000 rpm for 5 min and extracted NADH/NAD supernatant was transferred into another micro centrifuge tubes. Total NADt (NADH and NAD) and NADH were determined according to the protocol provided by NAD⁺/NADH Quantification Colorimetric Kit (BioVision, Milpitas, Calif., USA). Briefly, to measure total NADt (NADH and NAD), 50 μl of extracted protein samples were transferred into a 96-well plate. To determine NADH, 200 μl supernatant of extracted protein samples were heat decomposed at 60° C. for 30 min, cooled on ice, centrifuged and transferred 50 μl supernatant into labeled 96-well plate. Standard curve and reaction mix was prepared according to the manufacturer's protocol. Then developer was added in each well and plate was read spectrophotometrically at 450 nm following a 3 h incubation period. NAD/NADH ratio was calculated as: (NADt−NADH)/NADH.

Western blotting to determine alteration of acetylation and p-ADP-Rybosylation in B1Tg Peri-Cre heart proteins

Protein samples were prepared by homogenizing each control and experimental mice heart samples (˜20 mg) in 400 μl of 1X-SDS-PAGE loading buffer with PARP1 inhibitor (1:1000, provided by Dr. Mathew). Then cell lysate was sonicated twice for 10 sec each in ice and centrifuged at 15,000 rpm for 1 min. Supernatant was collected carefully and used for western blotting to determine alteration of acetylation and p-ADP-rybosylation in control and experimental B1Tg-PeriCre mice heart samples. Primary IgG antibodies for. Acetylation and pADPr were provided by Dr. Mathew and were used at a dilution of 1:1000 in 5% milk-TBST). The horseradish peroxidase conjugated anti-rabbit secondary IgG antibody (Cell Signaling) was used at 1:5000 dilution to detect a primary IgG antibody. Western blots were incubated with Clarity western ECL detection reagents (Bio-Rad laboratories) and exposed to X-OMAT AR films (Eastman Kodak. Rochester, N.Y., USA) for autoradiography. The autoradiograms were scanned on an EPSON Scanner using Photoshop software (Adobe Systems, Seattle, Wash., USA).

Tissue processing and preparation: Mice (P0-12 months) were euthanized using Isoflurane inhalation, hearts were perfused 0.9% saline solution, fixed in 4% paraformaldehyde-PBS solution. Tissue processing was performed as following: hearts were washed in phosphate buffer saline 1XPBS (3 changes X 15 minutes), dehydrated in gradient alcohol 70%, 95%,100% (3 changes X 2 hours), cleared in Xylene (3 changes X 3 hours), infiltrated with paraffin (3 changes X 4 hours), and final embedding in paraffin using tissue tack cassette Serial sections of the entire aortic valve region were obtained at 7 μm thickness using Leica microtome. In preparation for histological staining, tissue deparaffinization and rehydration was performed as following: sections were cleared in xylene (3 changes X 10 minutes), rehydrated in gradient alcohol 100%, 95%, 70%, and finally in water (2 changes X 5 minutes).

Histopathology: morphological changes were analyzed using hematoxylin and eosin stain (H&E) and pentachrome, trichrome and Alcian blue staining. H&E was performed as following: slides were stained in Gill's hematoxylin for 4 minutes, rinsed in running water for 5 minutes, dipped in Scott's solution for 1 minute to turn hematoxylin blue, dehydrated in 95% alcohol for 1 minutes, counterstained with Eosin for 1 minute, dehydrated in graded alcohol 70%, 95%,100% (2 changes X 5 minutes), cleared in Xylene (3 changes X 5 minutes) and cover slipped with permanent mounting media. Russel-Movat pentachrome staining (American Master Tech-catalog#) was performed according to the manufacture protocol: sections were first stained for 15 minutes in Verhoeffs elastic stain prepared freshly by mixing equal volume of 10% alcoholic, Absolute alcohol, 10% Ferric Chloride, and Universal Iodine. After washing in deionized water, slides were dipped in 2% Ferric Chloride for 1 minute then rinsed in deionized water. Slides then immersed in 5% Sodium Thiosulfate for 1 minute then rinsed in deionized water. Stain for mucins was performed by dipping slides in slide in 3% acetic acid followed immersion in 1% Alcian blue solution for 15 minutes then rinsed in deionized water. Slides incubated in Crocein Scarlet Acid Fuc for 2 minutes, rinsed in deionized water, dipped in 1% acetic acid, and connective tissue was differentiated by immersing slides in 5% Phosphotungstic acid for 4 minutes followed by dipping in 3′ Acetic acid. For collagen staining, slides were dipped in absolute alcohol for 2 minutes followed by immersion in Saffron solution for 15 minutes. Finally, slides where dehydrated in 100% ethanol, cleared in Xylene and cover slipped with permanent mounting media. Alcian blue staining (American Master Tech-catalog#) was performed as following: sections were immersed in 3% acetic acid for 3 minutes and stained in Alcian blue pH 2.5 for 30 minutes, washed in water for 1 minute, counterstained with nuclear fast red stain for minutes, dehydrated in gradient alcohol (70%, 95% 100%), cleared in Xylene (3 changes X 5 minutes), and cover slipped with permanent mounting media. Aortic valve leaflet surface area was measured using image pro plus software (REF): AOV tri-leaflets view were captured using Nikon optiphote microscope 4×objective for the male and female dtg and control groups (3 sections per animal 21 micron apart). Images were calibrated, and the trace tool was used to mark the aortic valve leaflets including the base for each section and the area per section in um² was measured. The average area of the 3 sections per animal (at least 3 animals per group were analyzed) were used for statistical analysis

Detection of AOV calcification: Calcium deposits were detected in AOV section using Alizarin Red and Von Kossa stains following manufacture protocol (American Master Tech). Alizarin S Red staining was performed as following: slides were immersed in Alizarin S Red solution for 5 minutes, acetone for 15 seconds, 1:1 Acetone/xylene for 15 seconds, followed by clearing in xylene (3 changes X 5 minutes). Von Kossa staining was performed as following: slides were incubated with 5% Silver Nitrate for 20 minutes under UV light, 5% Sodium thiosulfate for 3 minutes, rinsed in water for 1 minutes, counterstained with nuclear red stain for 5 minutes, dehydrated in alcohol (3 changes X 1 minutes), and cleared in xylene (3 changes X 5 minutes). Calcification was quantified using image pro plus software as following: AOV were captured using Nikon optiphote microscope 4×objective for the male and female dtg and control groups (3 sections per animal 21 micron apart). Images were calibrated, and the count tool-threshold tools were used to mark alizarin red positive area. The total calcification area per section in um² was measured and the average area of the 3 sections per animal (for at least 3 animals per group) were used for statistical analysis Quantification of AOV fibrosis: Collagen content (fibrosis) was detected using Micro-Sirius Red and Trichrome stains following manufacture protocol (American Master Tech). Micro-Sirius Red Alizarin S Red staining was performed as following: sections were stained with Weigert's hematoxylin solution for 5 minutes, rinsed in water for 2 minutes, incubated with Micro-Sirius Red stain for 1 hour, rinsed in 0.5% acetic acid (2 changes X 5 seconds), dehydrated in alcohol (3 changes X 10 seconds), cleared in xylene (3 changes X 5 minutes). AOV were captured using Nikon optiphote microscope 4×objective for the male and female dtg and control groups (3 sections per animal 21 micron apart). Images were calibrated, and the trace tool was used to mark the aortic valve leaflets including the base for each section. The count tool was used to quantify the area per um² and the average area of the 3 sections per animal (at least 3 animals per group were analyzed) were used for statistical analysis

TUNEL apoptosis: Apoptosis was detected using FragEL™ DNA Fragmentation Detection Kit, Colorimetric—TdT Enzyme Cat#QIA33, following manufacture protocol: sections were deparaffinized in xylene (3 changes×5 minutes), rehydrated through gradient alcohol concentrations 100%, 95%, 80%, and 70% (2 changes X 3 minutes) followed by immersion in 1XTBS (2 changes X 5 minutes). Sections were permeabilized using 1% Proteinase K in Tris buffer for 15 minutes at room temperature. After wash in 1×TBS, endogenous peroxidase was inhibited with 30% H₂O₂- Methanol (1:10 dilution) for 5 minutes at room temperature followed by immersion in 1X TdT Equilibration Buffer for 30 minutes at room temperature. To mark apoptotic cells, sections were incubated in TdT Labeling Reaction Mixture (contains the terminal deoxynucleotidyl transferase TdT Enzyme) in humidified incubator for 1 hour at 37° C. After 3 washes in 1×TBS, the reaction was terminated by immersion slides in stop buffer for 5 minutes followed by 3 washes in 1×TBS. To detect the signal, sections were immersed in blocking buffer for 5 minutes, incubated with 1X conjugate in blocking buffer for 30 minutes at room temperature, rinsed in 3 changes of 1XTBS, and incubated with freshly prepared DAB solution. Nuclei were counterstained with methyl green and cover slipped with Permount mounting media. Images were obtained using NIKON Optophote light microscope using 4× objective. Quantification of percent apoptosis per was performed using Image Pro Plus software as following: first an area of interest was drawn surrounding the OFT cushion and used constantly for all sections measured, the percent of apoptosis was averaged from 3 sections per animal with total of 3 experimental and 3 littermate control embryos using corresponding sections. Data were analyzed by Sigma plot software using student T test and a P value of 0.05 is considered significant

Immunohistochemistry:

Formalin Fixed Paraffin embedded tissue was cut into 7 pm thick slices. Sections were deparaffinized in three changes of xylene and rehydrated in graded concentrations of ethanol (100%, 95%, and 70%) followed by two washes in distal water. Heat Induced antigen retrieval was performed by incubation tissue sections in heated Target solution (citrate buffer) for 15 minutes, followed by blocking of endogenous peroxidase and alkaline phosphatase in dual enzyme block (dako) for 10 minutes. After washing, sections were incubated overnight at 4c in the Rabbit Anti-Hemagglutinin (HA Tag) Antibody diluted in DAKO antibody diluent. Primary antibodies was detected using Dako LSAB kit according to manufacture protocol in which tissue sections were incubated with universal biotylnated link for 30 minutes, HRP-conjugated streptavidin for 30 minutes and signal detection using diaminobenzidine (DAB) chromogen followed by counterstaining of the nuclei with hematoxylin. For Immunofluorescence, similar steps for deparffinization, rehydration and antigen retrieval were performed.

Serum Biochemical and ELISA Analysis:

Serum biochemistries (calcium, phosphorus, blood urine nitrogen (BUN), creatinine, and alkaline phosphatase) from 10-mo-old mice were measured using an RX Daytona clinical chemistry analyzer (Randox Laboratories).

Serum ELISA analysis was used to measure and compare the amount of TGFβ1 in age and gender matched control and Tgƒb1^{Tg};PostnCre mice according to published methods.

We describe a new developmental and cellular paradigm by which postnatal fusion of left and right coronary cusps of aortic valves, the raphe type, which is common in human BAV, underlies the pathogenesis of calcific AS in Tgƒb1 overexpressor mice. At molecular level, TGFβ1 hyperactivation resulted in increased interaction of activated SMAD3 with RUNX2 in aortic valves. In addition, TGFβ1 downregulated NAD+35-regulated poly-ADP(ribose)polymerase (PARP1)—and tyrosyl-tRNA synthetase (TyrRS)—dependent ADP-ribosylation of RUNX2, resulting in increased RUNX2-dependent osteoblast differentiation of valve interstitial cells.

Significantly, a direct TGFβ1 pathological effector function was verified using partial conditional genetic deletion of Tgƒb1, which prevented the calcific AS in Tgƒb1 transgenic mice. Together, our results show that TGFβ1 causes calcific AS through postnatal acquisition of BAV disease and that sex-based targeting of TGFβ1 signaling is a potential medical treatment for calcific aortic valve disease.

Increased TGFβ1 is found in human CAVD and pericardial fibrosis and calcification associated with pericarditis and TGFβ1 stimulates calcification in cells with osteoblastic potential. TGFβ1 can induce calcification in porcine and ovine valve interstitial cells (VIC). Despite the strong association between increased TGFβ1 and AV calcification, the role of TGFβ1 and its downstream mechanisms in the development and progression of CAVD in vivo remain unknown.

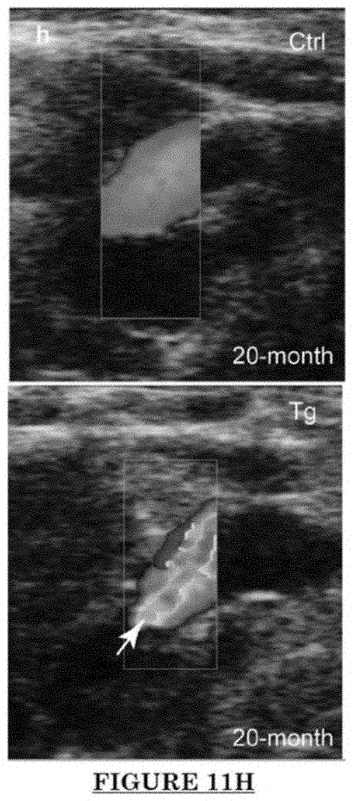

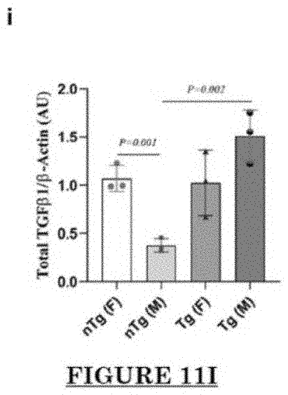

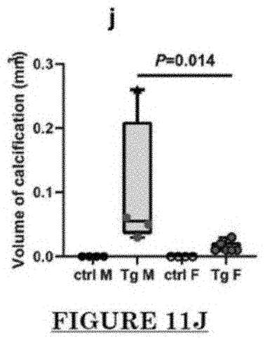

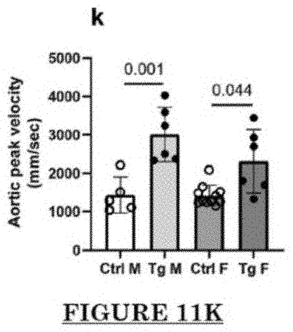

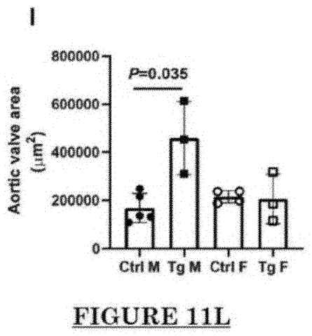

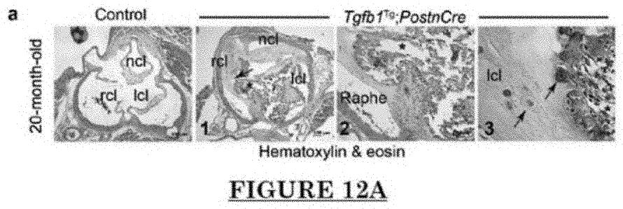

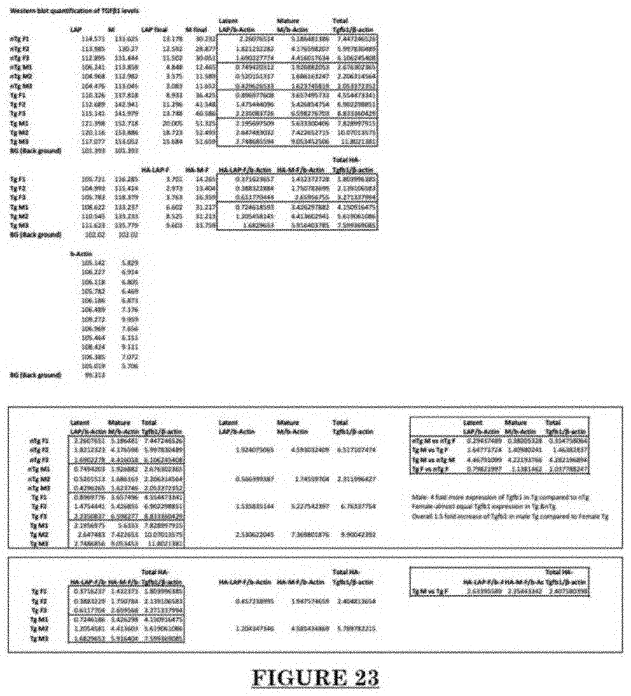

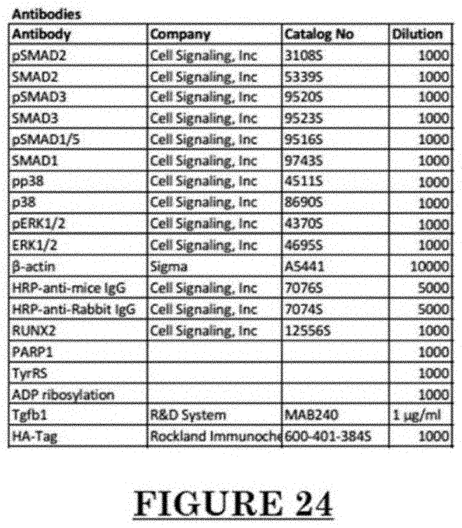

To determine if increased levels of TGFβ1 cause calcific AS, valve interstitial cell (VIC)—speck Tgƒb1 conditional transgenic (Tgƒb1^{TG};PostnCre) mice were produced by intercrossing the Tgƒb1 transgenic (Tgƒb1^{TG}) and Periostin-Cre (PostnCre) mice, see FIG. 11A. PostnCre mice drive Cre expression in cushion mesenchymal cells from E10 onwards in uteri) and in VIC of postnatal mice. In the Tgƒb1 transgene, the Tgƒb1 cDNA (hemagglutinin (HA) tagged) was mutated (Cysteine 223, 225 to Serine) to prevent the assembly of the latent associated peptide. This allowed direct binding of the secreted active dimeric TGFβ1 mature peptide to the TGFβ receptors. Western blotting of dissected AV using TGFβ1 and HA epitope-tagged antibodies confirmed the predominant ectopic expression of cleaved mature peptide and unprocessed pro-peptide of transgenically produced TGFβ1 (via anti-HA tag), and combined endogenously and transgenically produced mature and pro-peptides of TGFβ1 in both male and female Tgƒb1^{TG};PostnCre mice at 15 months of age (FIG. 11B and FIG. 23).

The levels of transgenically expressed HA-tagged total TGFβ1 (cleaved mature peptide+80 unprocessed pro-peptide) were 2.4 fold higher in AV from males than female transgenic mice. There was no HA-tagged TGFβ1 in control animals (FIG. 11B, bottom panel; i). Anti-TGFβ1 immunoblots, which did not distinguish between the endogenous and transgenic form of TGFβ1, indicated that there was a 4.3 fold increase in combined endogenously and transgenically produced total TGFβ1 in AV tissue from transgenic males compared to control male mice. Control male AV tissue had less total TGFβ1, than control female mice (FIG. 11B, i).

Intriguingly, there was no significant increase in total TGFβ1 in AV from female transgenic mice compared to control females because transgenic females compensated by producing less unprocessed pro-peptide of TGFβ1 (FIG. 11B, FIG. 23). Histological analysis revealed severe AV calcification and thickened AV in 15-20-month old male Tgƒb1^{TG};PostnCre mice (FIG. 11C). The female Tgƒb1^{TG};PostnCre mice developed mild CAVD, showed less AV calcification and AV thickening compared to the age-matched male Tgƒb1^{TG};PostnCre mice (FIG. 11E). Microcomputed tomography (microCT) confirmed the higher AV calcification in vivo in adult male compared to female Tgƒb1^{TG} 93;PostnCre mice (FIGS. 11D, 11F, 11J). AV calcification was predominantly seen in the right and left coronary leaflets, which were fused. The non-coronary AV leaflet was not affected in Tgƒb1^{TG};PostnCre mice up to the age of 24-months. Aortic valve hemodynamics, as assessed by echocardiography in a longitudinal study was consistent with the presence of AS in 20-month-old Tgƒb1^{TG};PostnCre mice (FIGS. 11G and 11H). The peak aortic jet velocity was markedly increased in both male and female Tgƒb1^{TG};Postncre mice compared to age and sex matched control mice (FIG. 11K).

The data showed that baseline cardiac parameters (such as left ventricular ejection fraction and fractional shortening) were significantly reduced in the select older Tgƒb1^{TG};PostnCre mice which developed hemodynamically significant stenosis calcific AS (FIGS. 11C, 11D, and 11K). Morphometric comparison revealed significant thickening of the AV in Tgƒb1^{TG};PostnCre mice (FIG. 11C, 1). Overall, these results closely model the clinical findings suggesting that CAVD affects more men than women at younger ages. Thus, our results establish that although increased levels of bioactive TGFβ1 spontaneously causes CAVD in both sexes, sustained TGFβ1 hyperactivation drives the progression to calcific AS in males.

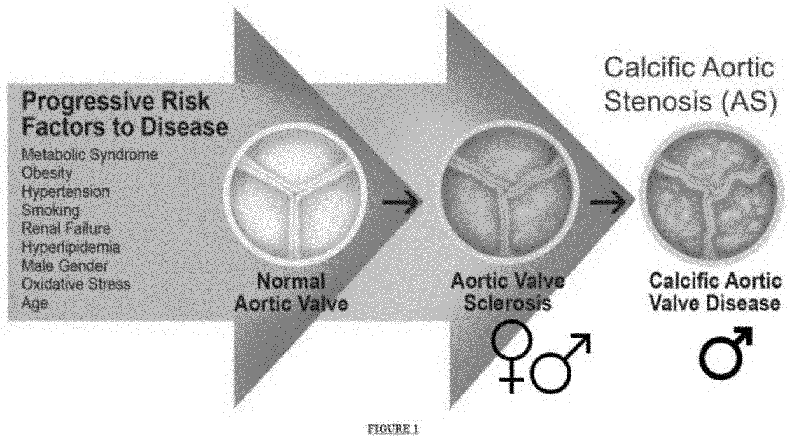

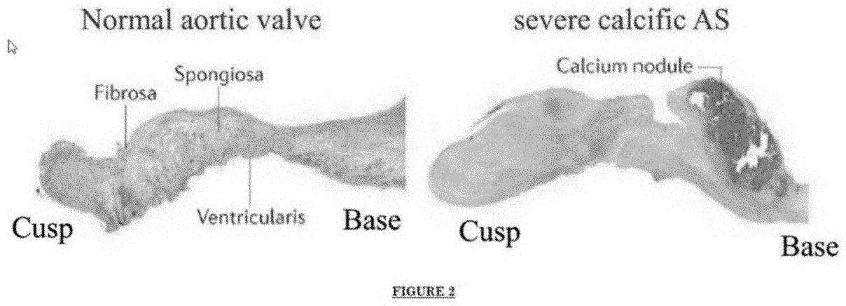

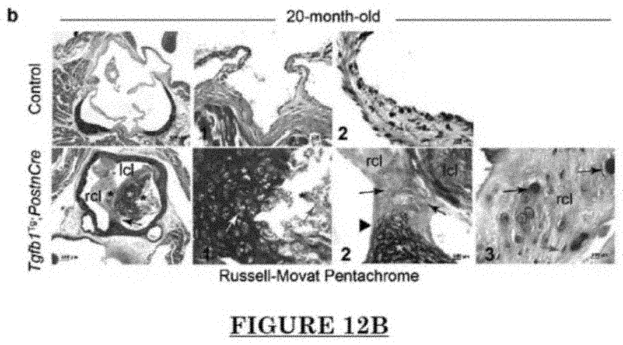

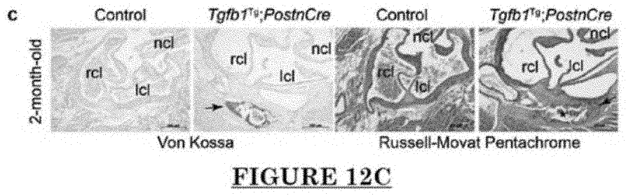

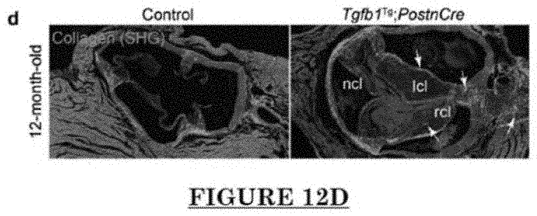

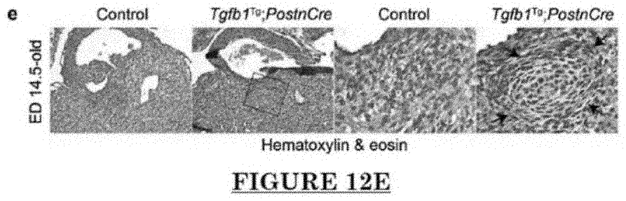

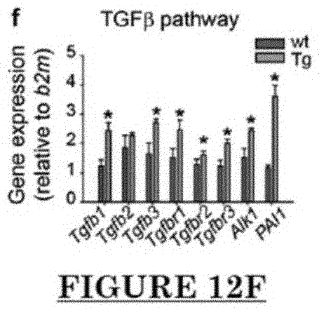

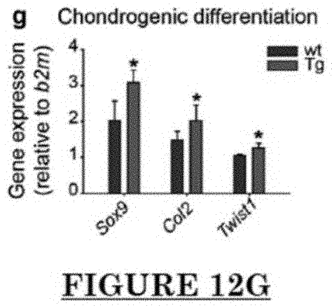

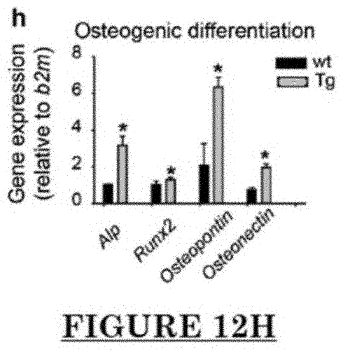

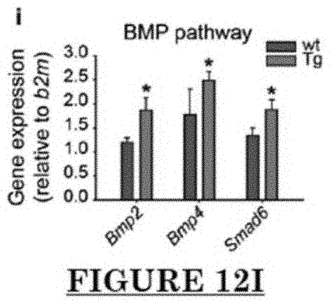

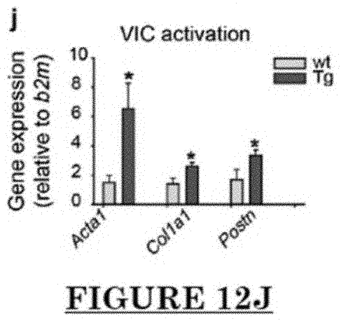

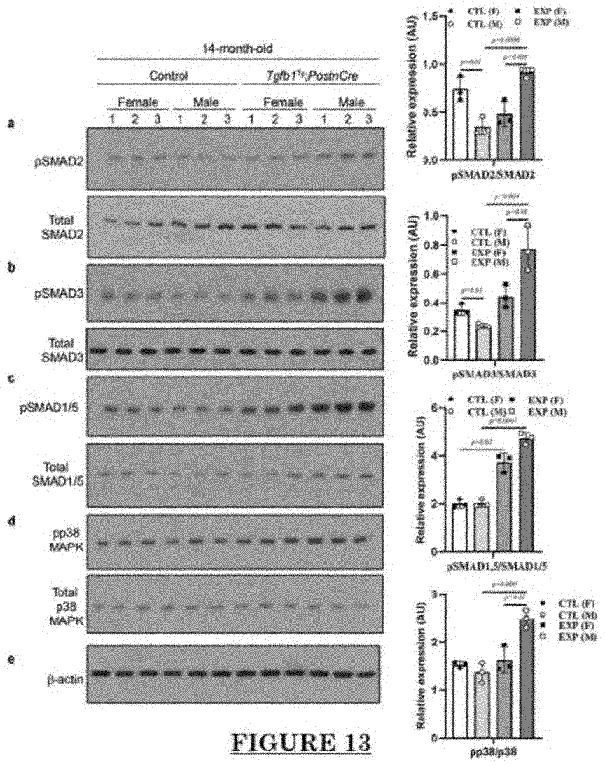

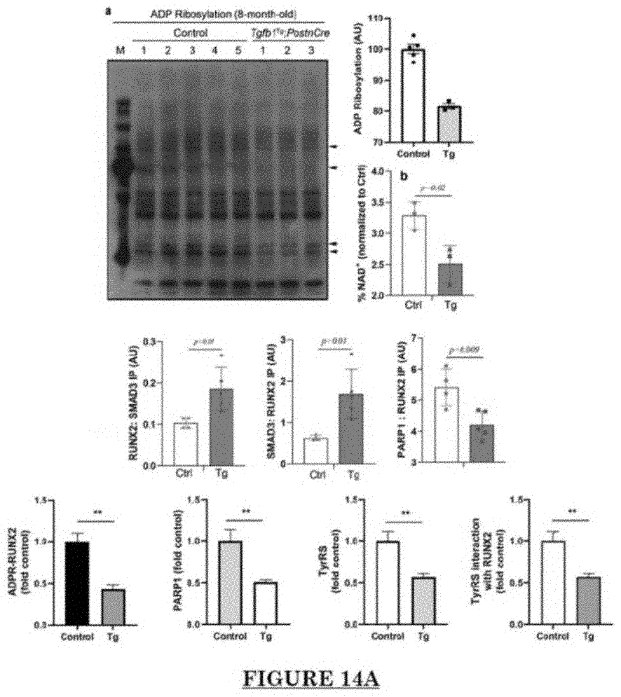

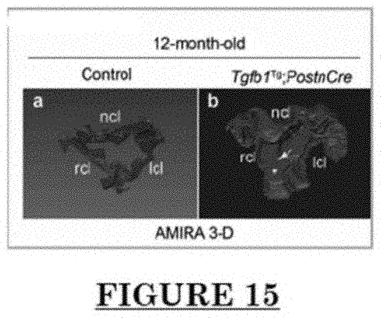

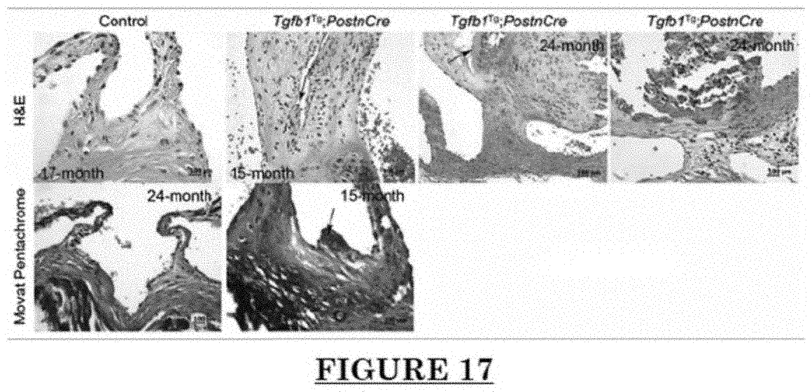

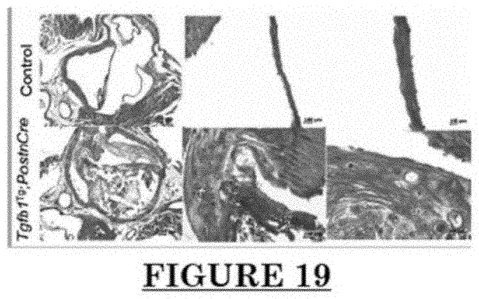

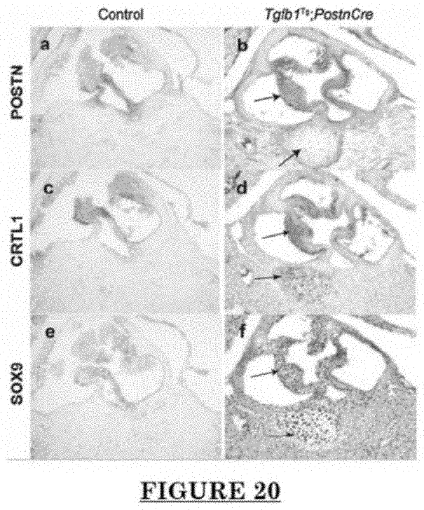

Importantly, histological studies showed that the right and left coronary cusps of the AV were predominantly fused at the base and formed a raphe resulting in a Type 1 BAV morphology in male Tgƒb1^{TG};PostnCre mice (FIG. 12A, FIG. 15). The overall length of the raphe (from annulus to separation of the cusps) increased with age and resulted in near complete fusion of right and left coronary cusps in the male Tgƒb1^{TG};PostnCre mice which developed symptomatic calcific AS, as confirmed by Doppler echocardiography and subsequent microCT and histological analysis. Histological analysis of disease progression at different ages revealed the emergence of a blood-filled cystic structure which fused with valve endothelial cells of the right and left AV cusps as it continuously grew larger and calcified while acting as zipper at the front end which zipped/fused both leaflets from base to cusps (FIG. 17). We also noticed that many of the blood clots seamlessly fused with the AV cusps, with the help of extensive collagen tracks between them, further contributing to the progression of aortic stenosis (FIG. 19). Histological analysis also revealed significant calcific deposits (i.e., via active endochondral bone formation) and the presence of cartilage in the aortic valve of Tgƒb1^{cTG};PostnCre mice (FIG. 1.2A). Activated valve interstitial cells in the male transgenic mice produced excess collagen 1 as dense connective tissue (i.e., served as perichondrium) and differentiated into chondroblasts, which became a continuous source of actively proliferating chondrocytes (i.e., isogenous groups) (FIG. 12B). These chondrocytes produced a significant amount of cartilage-specific matrix, hypertrophied, and died by apoptosis (FIG. 12G, FIG. 19). The empty interstitial space left behind by cell death was filled by bone-marrow-derived osteoprogenitor cells which recruited osteoblasts on to the calcified cartilage spicules, and via endochondral bone formation differentiated into osteocytes residing within the calcified collagen-rich bone matrix (FIGS. 12B and 12H). Masson's trichrome staining showed significant fibrosis and collagen accumulation in Tgƒb1^{cTG};PostnCre mice which increased as the diseased AV progressed to advanced fibrocalcific disease (FIGS. 12A, 12B, 12D, and FIG. 19).

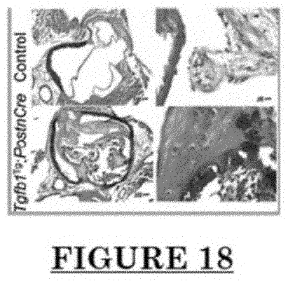

Gene expression analysis of dissected AV tissue from 4-month-old mice confirmed increased osteochondrogenic differentiation, TGFβ/BMP signaling, and valve interstitial cells (VIC) activation (FIGS. 12F-12J and FIG. 19). We also noted that nodular calcification of the AV cusp region in older males was associated with increased fragmented elastic fibers (FIG. 18). One distinguishing feature of CAVD in Tgƒb1^{TG};PostnCre mice was the asymmetric initiation and progression of calcification during disease progression. This AV calcification has a significant resemblance to the AV calcification reported in calcific AS patient. See, Gomez-Stations, M. V. et al. Calcification and extracellular matrix dysregulation in 492 human postmortem and surgical aortic valves. Heart 105, 1616-1621, doi:10.1136/heartjnl-2019-314879 (2019) and Yabusaki, K. et al. Quantification of Calcified Particles in Human Valve Tissue Reveals Asymmetry of Calcific Aortic Value Disease Development. Front Cardiovasc Med 3, 44, doi:10.3389/fcvm.2016.00044 (2016). Intrinsic calcification began at the base of the AV, near the attachment to the aortic wall in the aortic annulus and AV hinge region at 4-weeks of age (FIG. 12C). Histological and morphometric studies also revealed that all older female

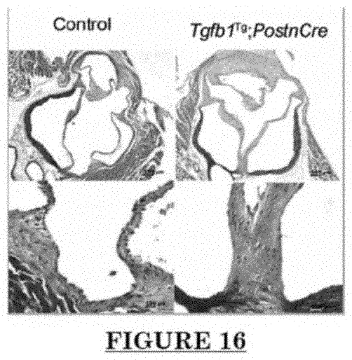

Tgƒb1^{TG};PostnCre mice had mild to moderate calcification, thickening of AV, and significant AV dysfunction but there was no progression to calcific AS up to 24-month of age (FIG. 16). Since collagen organization is important for AS30, we determined the collagen accumulation and organization by confocal laser scanning multi-photon microscopy via second harmonic generation and trichrome histochemistry. In control mice, collagen at the base of the cusps was oriented primarily parallel or perpendicular to the long axis of the attachment site (FIG. 12D). In Tgƒb1^{TG};PostnCre mice, there was excess and disarrayed mesh of collagen fibers at the base of the valve cusps, which tethered adjacent cusps to one another (FIG. 12D). Thus, although total collagen in the valve was increased in Tgƒb1T^{TG};PostnCre mice, collagen was remodeled at the base of the valve in Tgƒb1^{TG};PostnCre mice to form a mesh that spans adjacent cusps, which may restrict opening of the valve resulting in symptomatic aortic stenosis in males (FIGS. 11G-11H). In addition, trichrome staining confirmed significant age-dependent fibrosis of aortic leaflets, the aortic root, and the AV hinge regions of Tgƒb1^{TG};PostnCre (FIG. 19). Thus, although male transgenic mice were born with three AV leaflets the fusion of left and right coronary AV leaflets occurred over the course of a year with an incomplete raphe underlying the pathogenesis of calcific AS. Although PostnCre promoter is active in the three AV leaflets during cardiac outflow tract development, cardiac neural crest cells are primarily restricted to left and right coronary leaflet. This raises the possibility that both endocardial—and cardiac neural crest-derived valve interstitial cells are involved in left and right coronary cusps fusion and calcific AS. This finding suggests a new clinical paradigm that right and left coronary cusps fusion, predominantly reported by clinicians in the surgical specimens of individuals with calcific AS, can be an acquired Type 1 BAV phenotype and may not be a congenital BAV malformation as often anticipated in the current clinical practice.

To determine the developmental and cellular origin of the CAVD in adult mice, Tgƒb1^{TG};PostnCre mice were analyzed during heart development and postnatal stages (neonates, juvenile, adult). The data showed that autonomous PostnCre-derived endocardial cushion cells from embryonic day (E)14.5 embryonic hearts producing bioactive TGFβ1, trans-differentiated into a mesenchymal cell aggregate or nodule in the periaortic region (FIG. 12E). TUNEL, analysis indicated that Tgƒb1-overexpressing endocardial cushion cells escaped normal apoptosis, which occurs during normal outflow tract remodeling (FIG. 20).

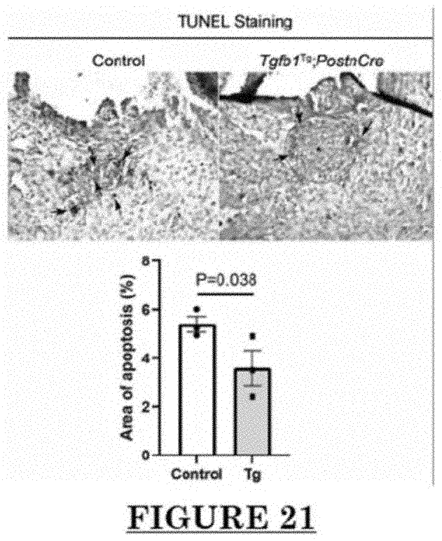

We identified that the calcific AS caused by TGFβ1 hyperactivation predominantly in the male Tgƒb1^{TG};PostnCre mice was mediated by increased activation of SMAD2. SMAD3, SMAD1/5 and p38 MAPK: and decreased activation of pERK1/2 MAPK signaling pathways in dissected AV tissues (FIG. 13, FIG. 21). Since increased bioactive TGFβ1 resulted in mild CAVD that did not progress to calcific AS in the female Tgƒb1^{TG};PostnCre mice (FIG. 11E, 11F, 11J, and FIG. 16), SMAD1/5 activation was increased and that there was no significant changes in levels of activated SMAD2, SMAD3, p38 MAPK and pERK1/2 MAPK in female Tgƒb1^{TG};PostnCre mice (FIGS. 11A-11E). Our results are supported by recent studies indicating that SMAD1/5 activation contributes to CAVD³³ and TGFβR1- and ALK2-mediated TGFβ signaling via both SMAD3 and SMAD1/5 pathways is essential for the full TGFβ-induced transcriptional program and physiological responses. See Ramachandran, A. et al. TGF-beta uses a novel mode of receptor activation to phosphorylate SMAD1/5 and induce epithelial-to-(nesenchymal, transition. Elife 7. doi:10.7554/eLife.31756 (2018). It is not clear why ERK1/2 MAPK was downregulated in male transgenic mice that developed both AV calcification and AS. Collectively, our data indicate that TGFβ1 signaling via increased SMAD2, SMAD3, SMAD1/5, p38 MAPK contributes to calcific AS in the males; whereas TGFβ1-induced activation of SMAD1/5 alone contributes to the AV calcification in females.

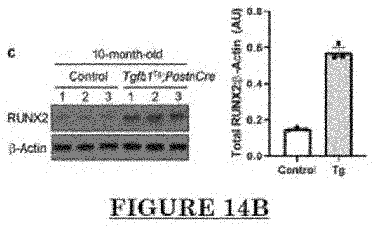

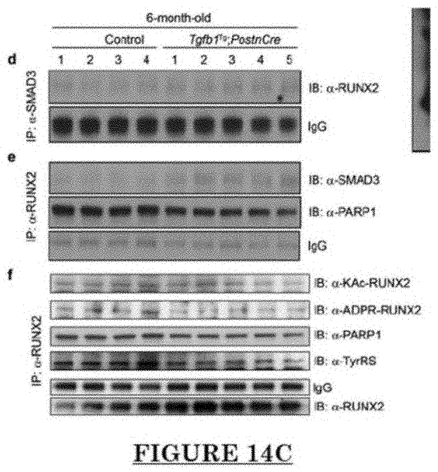

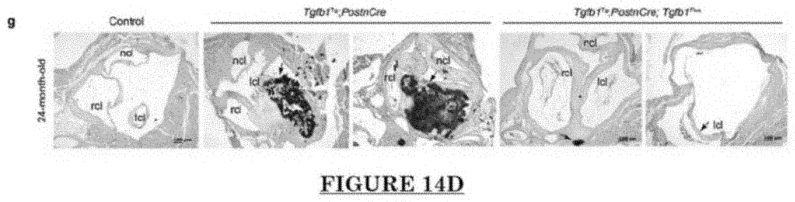

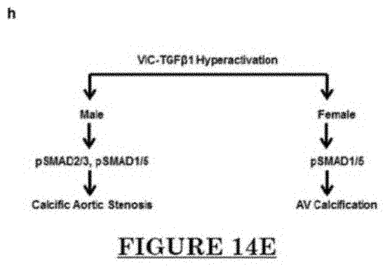

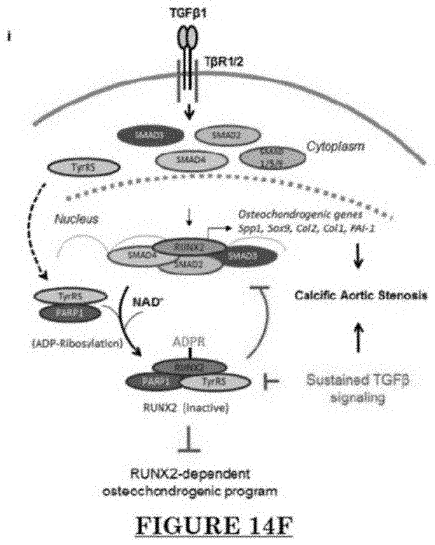

Next, we investigated TGFβ1-dependent molecular mechanisms that underlie calcific AS (see FIGS. 14A-14I.). The levels of total NAD+190 in cell lysate as well as poly-ADP-ribosylation were reduced in AV from Tgƒb1^{TG} 191;PostnCre mice (FIG. 14A). Biochemical data revealed an interaction of poly(ADP-ribose) polymerase 1 (PARP1) (also known as NAD*ADP-ribosyltransferase 1) with SMAD3 and RUNX2 in the AV of adult mice (FIGS. 14C and 14D)). RUNX2 is an important transcriptional factor involved in osteogenic differentiation in AV calcification. Levels of total RUNX2 were significantly increased in the AV tissue of the 10-month-old male Tgƒb1^{TG};PostnCre mice compared to age and sex-matched control animals (FIG. 14B). Because acetylation of RUNX2 is known to activate its osteogenic/calcification function, we tested if acetylation levels of RUNX2 were affected in the AV of transgenic mice but did not detect any significant change in the levels of acetylation (FIG. 14C). However, recently, poly-ADP-ribosylation of RUNX2 was shown as a post-translational modification. Immunoprecipitation studies revealed that SMAD3 physically interacted with RUNX2, tyrosyl-tRNA synthetase (TyrRS), and PARP1 (FIG. 14C). TyrRS is a potent DNA damage independent activator of PARP. See Sajish, M. & Schimmel, P. A human tRNA synthetase is a potent PARP1-activating effector target for resveratrol. Nature 519, 370-373, doi:10.1038/nature14028 (2015). Because total poly-ADP-ribosylation as well as levels of NAD⁺ in the cell lysate was reduced in AV from Tgƒb1^{TG};Postncre mice (FIG. 14A), we tested if RUNX2 was modified in AV tissue by TGFβ1 hyperactivation. As expected, we observed robust ADP-ribosylation of RUNX2 in control samples. However, we detected a significant decrease in the ADP-ribosylation status of RUNX2 in the Tgƒb1^{TG};PostnCre mice (FIGS. 14C and 14D), indicating that ADP ribosylation is a potential post-translational modification that inhibits the transcriptional activity and osteogenic function of RUNX2. This observation is consistent with previous reports that ADP-ribosylation inhibits transitional activation by directly preventing their binding to DNA. Because PARP1 is a major ADP-ribosylation factor, we tested if PARP1 interaction with RUNX2 is affected in the AV tissue in the Tgƒb1 transgenic mice. The data indicated a significant decrease in the physical interaction of PARP1 with RUNX2 (FIG. 14C). Although PARP1 is normally activated by DNA damage, recent work demonstrated that TyrRS is a potent DNA damage independent activator of PARP137 215. Because RUNX2 is robustly ADP-ribosylated in the normal AV tissues, we hypothesized that PARP1-dependent ADP-ribosylation of RUNX2 is facilitated through a DNA damage independent mechanism.

Consistent with our hypothesis, we found that TyrRS physically interacted with RUNX2 in the control AV tissue samples whereas there was a significant decrease in the physical interaction of RUNX2 with TyrRS by TGFβ1 hyperactivation in the Tgƒb1^{TG};PostnCre mice (FIG. 14C), suggesting that TyrRS facilitated downregulation of PARP1-dependent ADP-ribosylation of RUNX2 in the Tgƒb1T^{TG};PostnCre mice. Because female transgenic mice did not develop calcific AS, similar molecular studies were not done in females. Future experiments will verify if TyrRS targeting compounds would facilitate RUNX2 ADP-ribosylation and downregulate TGFβ1-hyperactivation-driven pathological events involved in calcific AS. Finally, superimposed heterozygous conditional deletion of Tgƒb1 (via Tgƒb1x mice), see Azhar, M. et al. Generation of mice with a conditional allele for transforming growth factor beta 1 gene. Genesis 47, 423-431 (2009), in valve interstitial cells of adult Tgƒb1^{TG};PostnCre mice significantly rescued the development of CAVD and blocked its progression to calcific AS in male Tgƒb1T^{TG};PostnCre;Tgƒb1^{Flox}/+(compound) mice compared to male Tgƒb1^{TG};PostnCre (transgenic alone) mice (FIG. 14D). Furthermore, partial genetic reduction of endogenously produced TGFβ1 by VIC in vivo also significantly attenuated right and left coronary cusps fusion, AV thickening, and AV calcification caused by VIC-specific TGFβ1 hyperactivation in male Tgƒb1^{TG} 232;PostnCre mice.

This disclosure establishes a causative role of TGFβ1 in the development and pathogenesis of CAVD that recapitulates both age and sex-specific etiologies associated with BAV (Type 1) and calcific AS in humans. These results establish a reliable and convenient adult mouse model that ‘spontaneously’ (without hypercholesterolemia, hyperlipidemia, diabetes, chronic kidney disease, ANGII-infusion) develops calcific AS. These mice will be useful in developing and testing sex-specific diagnosis, prevention and treatment strategies for the CAVD. Currently, there are no mouse models, which could effectively recapitulate these features. The current available mouse models only describe a fusion between the right and non-coronary cusp (Type 2) with very little or no AV calcification, which is a clinically less prevalent type of raphe seen in calcific AS. Whereas in this study we describe a model with fusion of the right and left coronary cusps (Type 1) in males, the raphe type commonly observed in human BAVs. Why the raphe formation and calcific AS progression is seen in males and not females is being currently investigated. Our results have also uncovered novel developmental, cellular, molecular, and biochemical mechanisms and approaches for sex-specific therapeutic targeting to block the development and/progression of CAVD in mice (FIG. 14E). Novel observations are likely to emerge from these findings which will favorably shift the current paradigm and approaches for an early detection and prevention strategies for targeting specific pathways for a potential medical treatment of CAVD as well as calcific AS progression in older humans. AV calcification is a strong determinant of AS severity and powerful risk factor for overall cardiovascular-dependent mortality. Collectively, through these basic translational studies our results indicate that selective therapeutic partial reduction of TGFβ1 is an important sex-adapted strategy to attenuate AV calcification and the development and progression of CAVD and calcific AS.

Methods

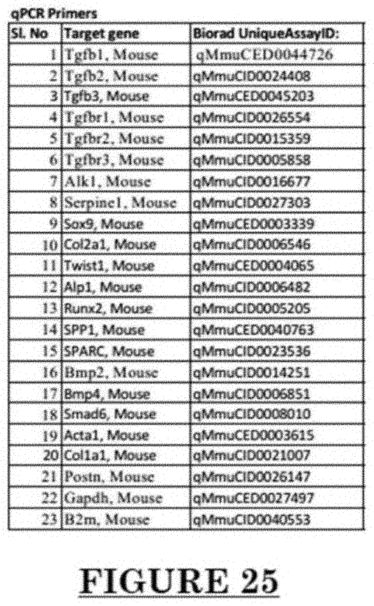

Mice and ethical statement: All animal procedures were performed in accordance to the approved authorized user protocol by the Institutional Animal Care and Use Committee (IACUC) and the University of South Carolina. Mice were euthanized using a procedure approved by the IACUC. Tgƒb1^{TG}; PostnCre mice and littermate controls were generated by mating Tgƒb1^{TG} female mice with PostnCre male mice. Tgƒb1^{flox} mice were used to generate Tgƒb1^{TG}; PostnCre;Tgƒb1^{+Flox} mice. In the transgenic mice, Tgƒb1 cDNA contained a fully bioactive hemagglutinin (HA) epitope tag which distinguished the transgenic bioactive TGFβ1 from the endogenous TGFβ1. The Tel transgene expression requires Cre-mediated deletion of an intervening floxed-enhanced green fluorescent protein (EGFP) gene in order for an ubiquitous β-actin promoter to transcribe constitutively active HA tagged Tgƒb1 cDNA. PostnCre mice express the Cre recombinase predominantly in the cardiac outflow tract endocardial cushion cells (E10.0) and adult VIC. Genotyping was performed using the standard protocol for DNA extraction and purification of tail tissue followed by polymerase chain reaction to identify Tel and Cre tranegenes using published primers and procedures, see FIG. 25.

Echocardiography:

Cardiac function, aortic root diameter, and ascending aortic diameters were measured noninvasively using a small-animal ultrasound and compared to age-matched controls. A high-frequency ultrasound device (Vevo 3100, Visual Sonics) with a linear array probe (MS 400D, frequency 30-55 MHz) was used for in vivo imaging of the aorta on isoflurane-anesthetized mice. Anesthesia was administered starting at 1% inhalation and adjusted up to 3% until a stable state was reached as indicated by a heart rate between 450 to 500 beats per minute and respiration between 50 to 100 breaths per minute. The body temperature was maintained at 37 degrees Celsius.

The aortic arch view was used to consistently get images of the aorta from the same anatomical direction. This was achieved by angling the right side of the platform downward. In B-mode and parallel to the body, the transducer was placed on the right side of the mouse's chest. Then it was slightly rotated clockwise until the aortic arch was in view. Videos and images were captured using the ECG-Gated Kilohertz Visualization mode. Peak AV flow velocity was captured with a combination of color and pulsed wave Doppler mode. The parasternal short axis view of left ventricle was obtained by angling the bottom of the platform downwards and slightly to the left. While in B-mode, the transducer was place in the parasternal position and rotated clockwise until the papillary muscles were in view. In M-mode the left ventricular end-diastolic dimension (LVFDD), left ventricular end-systolic dimension (LVESD), the anterior wall (AW), and the posterior wall (PW) thickness were measured. From these measurements the ejection fraction and the fractional shortening were calculated. Overall, we followed the published standard procedures to determine cardiac and valvular dysfunction in mice, see Chu, Y. et al. Fibrotic Aortic Valve. Stenosis in Hypercholesterolemic/Hypertensive. Mice. Arterioscler Thromb Vasc Biol 36, 466-474, doi:10.1161/ATVBAHA.1.15.306912 (201.6), Fang, M., Alfieri, C. M., Hulin, A., Conway. S. J. & Yutzey, K. E. Loss of beta-Catenin Promotes Chondrogenic Differentiation of Aortic Valve Interstitial Cells. Arterioscler. Thromb. Vasc. Biol, ATVBAHA (2014) and Casaclang-Verzosa, G., Enriquez-Sarano, M., Villaraga, H. R. & Miller, J. D. Echocardiographic Approaches and Protocols for Comprehensive Phenotypic Characterization of Valvular Heart Disease in Mice. J Vis Exp, doi:10.3791/54110 (2017).

Microcomputed Tomography (microCT):

Mice were euthanized at 18 months and hearts were dissected, fixed (4% paraformaldehyde) and collected in 1× phosphate buffered saline (PBS) for analysis. MicroCT scan images were acquired using Quantom GX micro CT imaging system (Perkin Elmer). All scans were taken with a standard filter (Cu0.06+Al0.5) with the voxel size of 36 um at 90 KV and 88 uA and were HU calibrated. Using Fiji software (NIH) all scans were reconstructed to a 3D image. To quantify calcification, the threshold was set to 120-255 and the voxels containing thresholded regions of the scan were counted using a voxel counter plug-in for Fiji. Knowing the voxel size, threshold volume was calculated. Overall, we followed the published method to quantify calcification volume in mice.

Histology:

Postnatal mice (up to 25-month-old) were euthanized and their hearts perfused with 1×PBS and 4% paraformaldehyde-PBS solution. Tissues were processed, embedded, and sectioned (7 μm) using published procedures. Histological analyses were done by Alizarin red, Von Kossa, Hematoxylin and Eosin (H&E), Russel Movat's Pentachrome, Masson's Trichrome, Alcian Blue, and Verhoeff Elastic stain kits. All staining was performed according to the manufacturer's (American MasterTech, Inc) protocols, and sections were mounted using permanent mounting media (Vector Lab). Aortic valve leaflet surface area (μm²) was measured using imagePro plus software according to published methods. The average area of the 3 sections per animal (at least 3 animals per sex and genotype group) was used for statistical analysis. All sections were visualized and photographed under brightfield optics on the Nikon Optiphot-2 (equipped with AxioCam MRc Camera) and EVOS TM FL Auto Imaging System (Thermaisher, Inc).

Immunohistochemistry:

Immunohistochemistry was performed according to published methods. Paraformaldehyde (4%)—fixed paraffin sections (7 μm) were deparaffinized and heat-induced antigen retrieval was performed by incubating the tissue sections in heated Target Retrieval Solution (DAKO USA) for 15-30 minutes, followed by blocking of endogenous peroxidase in Dual Enzyme Block (DAKO, USA) for 10 minutes. After washing, sections were incubated overnight with primary antibodies at 4° C. Primary antibodies were detected using an LSAB2 kit according to the manufacturer's protocol. Signal detection was done using diaminobenzidine (DAB) chromogen (DAKO) followed by counterstaining of the nuclei with hematoxylin. For Immunofluorescence, similar steps for deparaffinization, rehydration and antigen retrieval were performed. Fluorescently-labeled secondary antibodies (Invitrogen) were used to detect proteins of interest. All sections were visualized and photographed under brightfield optics on the Nikon Optiphot-2 (equipped with AxioCam MRc Camera) and/or EVOS TM FL Auto Imaging System (ThermoFisher, Inc). For photographing the fluorescence, images were illuminated with appropriate wavelengths, the Zeiss Axiovert 200 (equipped with Axiocam 503 color camera and ZEN 2.3 SP1 Imaging software) fluorescence microscope was used.

TUNEL Apoptosis:

Cell death was quantified in 4% paraformaldehyde-fixed paraffin sections of E13.5-14.5 mouse embryos using FragEL™ DNA Fragmentation Detection Kit (Colorimetric—TdT Enzyme Cat#QIA33), following the manufacture protocol (Sigma/Millipore). Sections were permeabilized using 1% proteinase K in Tris buffer for 15 minutes at room temperature before staining. Nuclei were counterstained with methyl green after the DAB staining and mounted the slides in permount mounting media (Vector Lab). Images were obtained using Nikon Optophot light microscope. Quantification of apoptosis (%) was performed using Image Pro Plus software. Regions of interest (ROI) were drawn in the outflow tract cushions and this ROI was used in all other subsequent sections. Average apoptosis (%) was calculated in corresponding sections from 3 sections per embryo in 3 experimental and 3 littermate control embryos.

RNA Isolation, cDNA Synthesis, and Quantitative PCR:

mRNA was isolated using Trizol (Invitrogen) and miRNeasy micro kit (Qiagen) according to manufacturer's protocols and eDNA was generated from 500 ng mRNA using Invitrogen kit according to manufacturer's instructions (Invitrogen) 500 ng cDNA was subject to quantitative PCR amplification (Biorad-CFX) using pre-validated gene specific primers procured from the vendor (Biorad Inc). Following PCR analyses, the cycle count threshold (Ct) was normalized to species specific housekeeping genes (GAPDH, B2M; purchased from Biorad) and the ΔCt and fold changes in experimental samples over controls was determined. Statistically significant differences in gene expression levels were determined using Student's t-test or one-way ANOVA plus a post-hoc test as indicated in the figure legend, on at least 3 independent experiments with p<0.05 considered significant.

Western blotting: Western blotting was performed with heart and aorta samples from different control and experimental mice. The tissue samples were weighed, cut into small pieces, and homogenized using Wheaton tapered tissue grinders (Thermo Scientific, Rockford. IL, USA) in M-PER mammalian protein extraction reagent (Thermo Scientific) with complete mini protease inhibitor cocktail (Sigma). Following homogenization lysate was subjected to brief sonication for 20 sec in ice and kept at room temperature for 20 mins. Then centrifugation was performed at 15,000 rpm for 20 min at 4° C. and the supernatants were collected. The total protein concentration in the supernatant was determined using Pierce BCA protein assay kit (Thermo Scientific, Rockford, Ill., USA) and the samples were stored at −80° C. until further use. Western blotting was performed, as described previously, using these protein samples and the primary IgG antibodies against pSMAD2, SMAD2, 364 pSMAD1/5, SMAD1/5, pp38, p38, pTAK1, TAK1, pJNK, JNK, pERK1/2, ERK1/2 etc. at a dilution of 1:1000. Primary IgG antibodies against all these proteins were procured from Cell Signaling (Cell Signaling Technology, Inc., Danvers, Mass., USA). The horseradish peroxidase conjugated anti-mouse or anti-rabbit secondary IgG antibody (Cell Signaling) was used at 1:5000 dilution to detect a primary IgG antibody. Western blots were incubated with Clarity western ECL detection reagents (Bio-Rad laboratories) and exposed to X-OMAT AR films (Eastman Kodak, Rochester, N.Y., USA) for autoradiography. The autoradiograms were scanned on an EPSON Scanner using Photoshop software (Adobe Systems, Seattle, Wash., USA). β-actin, clone AC-15 monoclonal primary anti-body (Sigma-Aldrich, St. Louis, Mo., USA) was used as loading control to compare equal loading in the SDS-PAGE.

Acetylation and ADP-Ribosylation Studies:

Protein samples were prepared by 376 homogenizing each control and experimental mouse heart sample (˜20 mg) in 400 μl of 1×-SDS-PAGE loading buffer with PARP1 inhibitor (1:1000, provided by Dr. Mathew). Then cell lysate was sonicated twice for 10 sec each in ice and centrifuged at 15,000 rpm for 1 min. Supernatant was collected carefully and used for western blotting to determine alteration of acetylation and p-ADP-rybosylation in control and experimental B1Tg-PeriCre mouse heart samples. Primary IgG antibodies for Acetylation and pADPr were used at a dilution of 1:1000 in 5% milk-TBST). The horseradish peroxidase conjugated anti-rabbit secondary IgG antibody (Cell Signaling) was used at 1:5000 dilution to detect a primary IgG antibody. Western blots were incubated with Clarity western ECL detection reagents (Bio-Rad laboratories) and exposed to X-OMAT AR films (Eastman Kodak, Rochester, N.Y., USA) for autoradiography. The autoradiograms were scanned on an EPSON Scanner using Photoshop software (Adobe Systems, Seattle, Wash., USA).

NAD⁺/NADH Quantification:

Dissected AV tissues from the mouse hearts were washed in cold PBS and homogenized in 400 μl of NADH/NAD extraction Buffer (BioVision, Milpitas, Calif., USA) in a Wheaton tapered tissue grinder (Thermo Scientific, Rockford, Ill., USA). Then tissue homogenates were centrifuged at 14000 rpm for 5 min and extracted NADH/NAD supernatant was transferred into another micro centrifuge tubes. Total NADt (NADH and NAD) and NADH were determined according to the protocol provided by NAD*/NADH Quantification Colorimetric Kit (BioVision, Milpitas, Calif., USA). Briefly, to measure total NADt (NADH and NAD), 50 μl of extracted protein samples were transferred into a 96-well plate.

To determine NADH, 200 μl supernatant of extracted protein samples were heat decomposed at 60° C. for 30 min, cooled on ice, centrifuged and transferred 50 μl supernatant into labeled 96-well plate. Standard curve and reaction mix were prepared according to the manufacturer's protocol. Then developer was added in each well and plate was read spectrophotometrically at 450 nm following a 3 h incubation period. NAD/NADH ratio was calculated as: (NADt−NADH)/NADH.

Figure Legends:

FIGS. 11A-11L: Sex-specific differences in the development and pathophysiology of calcific aortic stenosis caused by valve interstitial cell-specific TGFβ1 hyper-activation. 11A—generation of VIC-specific Tgƒb1 transgenic mice. 11B-Western blot analysis of dissected AV from 15-month-old mice showing cleaved, mature peptide (m) and unprocessed, pro-peptide (p) from endogenous TGFβ1 and transgenic hemagglutinin (HA) epitope tagged constitutively active form of TGFβ1. 11C and 11E—Alizarin red staining of heart cross sections from control (left panel) and 2 independent Tgƒb1^{TG};PostnCre (middle and right panels) mice to evaluate AV calcification. 11D and 11F—Micro-CT scans of the heart to evaluate the extent of AV calcification were obtained from 15-20-months-old mice. Representative coronal z-stacked images of the heart with the pathological AV calcification seen as radio-dense lesions (indicated by the white arrow) in the surrounding soft cardiac muscle tissue. 11G—Doppler display of peak velocity across the normal and stenotic aortic valve was measured at 1.5 m/s for control (g, top) and 3.5 m/s for transgenic (g, bottom) mice, respectively. 31H—Color Doppler interrogation showed a mosaic-color jet at the aortic valve during systole in transgenic mice (highlighted by an arrow). 11I-Quantification of total TGFβ1. Data are mean±s.e.m. P-values and individual values are given on the bar graph. Two-sided Student's t-tests (without corrections for multiple comparisons) were used. 11J—Quantification of ectopic AV calcification from micro-CT images. Data are 590 mean±s.e.m. P-values and individual values are given on the bar graph. Value from the oldest transgenic male (20 months of age) indicated by a solid triangle is excluded from the analysis. Unpaired Two-sided Student's t-tests (without corrections for multiple comparisons) were used. 11K—Quantification of AV peak velocity. P-values are indicated. Two-sided t-test (with Welch's correction) was used. 11L—Quantification of AV area. Data are mean±s.e.m. Two-sided Mann Whitney test with Exact P-value was used, and the significant P-value is indicated. Scale bars, 100 μm (11C and 11E).

FIGS. 12A-12J—Developmental origin of calcific aortic valve stenosis. 12A-H&E staining of heart cross sections of control male (left panel) and Tgƒb1^{TG} 598;PostnCre (2 and 3 are magnified images of 1) male mice showing the fusion (arrow, image 1) of right (rc1) and left (1c1) coronary leaflets. The non-coronary leaflet (nc1) was not fused but thickened. Asterisks indicate calcified regions of the right and left coronary cusps (image 2). Black arrows indicate chondrocytes and blue arrow points to an osteocyte (image 3). 12B—Russel-Movat Pentachrome staining of heart cross sections from male control (left panel, low magnification; right, high magnification images, 2) and male Tgƒb1^{TG};PostnCre (1-3, magnified images) mice show cartilage/cartilage matrix (yellow asterisks) and calcified nodule (black asterisks) in the AV. Isogenous clusters of proliferating and hypertrophied chondrocytes (white arrow, image 1) and osteoblasts are indicated (blue arrow, image 2). Magnified image 2 shows cartilage (white arrow) and the calcified region of the AV. Blue-green color indicates proteoglycan-rich cartilage matrix and the yellow color indicates collagen-rich bone matrix. Image 2 shows a well-formed raphe (arrowhead) with elastic fibers (purple, white asterisk). Black arrows depict numerous immature chondrocytes or chondroblasts approaching the calcified nodules in the right and left coronary cusps. Image 3 indicates numerous chondrocytes producing excessive cartilage proteoglycans (black arrows) and few remaining activated valve interstitial fibroblasts (blue arrow). 12C—Von Kossa (left 2 panels) and Russel-Movat pentachrome (right 2 panels) staining of heart cross sections from control and Tgƒb1^{TG};PostnCre mice to illustrate AV calcification and cartilage formation at 2 months of age. Von Kossa staining indicates significant calcification in the AV hinge region of the right and left coronary leaflets (black arrow). Pentachrome staining revealed extensive cartilage formation (arrow) and collagen (yellow staining). Asterisk indicates the lumen of calcific nodule. 12D—Representative cross-sectional multiphoton images of control and Tgƒb1^{TG};PostnCre mice at 12-months of age. SHG channel (green) represents the collagen content of the AV leaflets, raphe, and the hinge region. Note the excessive amount of collagen in the transgenic heart (arrows). The organization and orientation of the collagen fibers was also impaired, particularly in the raphe of the right and left coronary leaflets. 12E—H&E staining to show intra-cardiac condensed mesenchymal cellular nodules in the AV-hinge region (arrows, magnified image of the boxed region) in the Tgƒb1^{TG};PostnCre embryos (E14.5). 12F-12J, Analysis of gene expression markers in microdissected tissue from the AV region by qPCR in 4-month-old control and Tgƒb1^{TG};PostnCre mice. Data are organized around markers for TGFβ 628 signaling pathway (Tgƒb1, Tgƒb2, Tgƒb3, Tgƒbr1, Tgƒbr2, Tgƒbr3, Alk1, Pai1), cartilage differentiation (Sox9, Col2, Twist1), osteoblast differentiation (Alp1, Runx2, osteopontin (Spp1), osteonectin), BMP signaling pathway (Bmp2, Bmp4, Smad6), and valve interstitial cells activation or myofibroblast differentiation (α-smooth muscle actin (Acta1), collagen 1a1., periostin (Postn)). Three biological replicates (n=3) each evaluated for two experimental replicates. Quantitative data are shown as mean±s.e.m. Two-sided Student's t-tests (unpaired) were used. Significant comparisons are indicated by asterisk (P<0.05). Scale bars, 100 μm (12A, 12B, 12E), 200 μm (12C).

FIG. 13 Sex-specific differences in TGFβ signaling underlies the pathogenesis of calcific aortic valve stenosis, a-e, Western blots of 14-month-old control female (n=3), control male (n=3), Tgƒb1^{TG};PostnCre female (n=3) and Tgƒb1^{TG};PostnCre male (n=3) showing changes of phosphorylated and total proteins in canonical (pSMAD2/SMAD2, pSMAD3/SMAD3, pSMAD1/5/9/SMA.D1/5) and non-canonical (pp38 MAPK/p38 MAPK) TGFβ signaling pathways in microdissected AV tissue. β-actin was used as a loading control. Data are mean:±s.e.m. P-values and individual values are indicated. Unpaired Two-sided Student's t-tests (without corrections for multiple comparisons) were used.

FIGS. 14A-14F—Molecular mechanisms—The SMAD3, which is activated by TGFβ1 signaling, physically interacts with RUNX2, PARP1, and TyrRS, and activated RUNX2 via post-translational modification by decreasing TyrRS-PARP1-dependent ADP ribosylation of RUNX2. FIG. 14A—Western blot of control and Tgƒb1^{TG} 647;PostnCre mice showing post-translation modification (acetylation, ADP ribosylation) in the microdissected AV tissue. 14A—Total NAD⁺648 content in microdissected AV tissue samples from control and Tgƒb1^{TG};PostnCre mice were compared using a commercially available BioVision NAD⁺650/NADH quantitation colorimetric kit 14CB and 14C—Western blots of control and Tgƒb1^{TG};PostnCre male mice showing RUNX2 and its physical interaction with SMAD3, PARP1, and TyrRS in the microdissected AV tissue. Data are mean±s.e.m. P-values and individual values are indicated. **P<0.005. Unpaired Two-sided Student's t-tests (without corrections for multiple comparisons) were used. 14D—Alizarin red staining of heart cross sections from 24-month-old control (left panel), 2 independent Tgƒb1T(=;PostnCre (1 middle panels), and Tgƒb1^{TG};PostnCre:Tgƒb1^{+/Flox} (2 right panels) mice to evaluate AV calcification. Note that AV calcification and the fusion of right and left coronary cusps was significantly rescued in Tgƒb1^{TG};PostnCre;Tgƒb1 ^{+/flox} mice (2 right panels) compared to Tgƒb1^{TG};PostnCre (2 middle panels). Scale bars, 100 μm. 14E and 14F-Models illustrating the TGFβ1-dependent molecular pathways involved in AV calcification in females and calcific AS in males, and mechanism of TGFβ1/SMAD3 mediated TyrRS/PARP1 interaction with RUNX2 in the 662 pathogenesis of calcific aortic valve stenosis.

FIG. 15—Analysis of AV leaflets fusion in male VIC-Tgƒb1^{TG} mice by 3 D morphological reconstruction. α-b, H&E stained serial sections of heart cross sections of control male (a) and Tgƒb1^{TG};PostnCre (b) are reconstructed using AMIRA computer software. Fusion of right coronary leaflet (rc1) and left coronary leftlet (1c1) in transgenic mice is shown by arrow (b). Asterisk indicates raphe.

FIG. 16—Histological analysis of raphe formation in female VIC-Tgƒb1^{TG} mice. Russel-Movat Pentachrome staining of heart cross sections of female control (left panel: low magnification, top: high magnification, bottom) and female Tgibra 696;PostnCre (right panel: low magnification, top; high magnification, bottom) mice showing AV thickening and raphe formation.

FIG. 17—Zipper model of right and left AV leaflets fusion and subsequent AV calcification in the pathogenesis of calcific aortic valve stenosis, a, H&E staining (top row) and Movat pentachrome (bottom row) staining of heart cross sections of control male and Tgƒb1^{TG};PostnCre mice showing a cystic protrusion of a blood filled structure at a younger age (15-months) (arrows) which acted as a puller and zipped the two leaflets (base to tip region) and transformed into a calcific nodule at later ages (24-months) (arrows, top, middle and right H&E-stained images). This cystic structure connects the ‘intrinsic’ calcification of the AV hinge region to more advanced ‘nodular’ calcification of the AV cusp region.

FIG. 18—Increased elastin fragmentation in advanced nodular calcification of the aortic valve cusp region during calcific AS progression. Verhoeff Elastic staining of heart cross sections of 20-month-old control male (top) and Tgƒb1^{TG};PostnCre (bottom) mice showing increased fragmented elastin associated with AV cusp calcification in the transgenic mice. Right panels are magnified images of the left panels.

FIG. 19—Fibrocalcific disease in stenotic AV valves of VIC-specific Tgƒb1; transgenic mice. Masson's trichrome staining of heart cross sections of 20-month-old control male (top) and Tgƒb1^{TG};PostnCre (bottom) mice showing increased collagen fibers accumulation in the AV hinge and cusp region in the transgenic mice. The right coronary cusp region fused seamlessly with an adjacent blood clot with collagen tracks contributing further to aortic valve stenosis in the transgenic mice (bottom, middle panel). Right panels are magnified images of the left panels.

FIG. 20—Increased osteochondrogenic proteins are associated with CAVD in the VIC-specific Tgƒb1 transgenic mice, a-f, Immunohistochemistry of heart cross sections of 2-month-old control (a, c, e) and Tgƒb1^{TG} 721;PostnCre (b, d, f) mice showing increased expression of periostin (α-b), cartilage-link protein (c-d), and SOX9 (e-f) in the AV region of VIC-Tgƒb1 transgenic mice.

FIG. 21—TGFβ1 hyperactivation in cushion mesenchymal cells inhibits their apoptosis during heart development. TUNEL staining of embryonic heart cross sections of E13.5 control (left) and Tgƒb1^{TG};PostnCre (right) embryos showing decreased apoptosis of the outflow tract cushion mesenchymal cells resulting in mesenchymal cell condensation and nodule formation. These cells later differentiate as cartilage and, over time, contribute to calcific aortic valve stenosis. Data are mean±s.e.m. Unpaired one-sided Student's t-tests were used. P-values and individual values are indicated. **P<0.038.

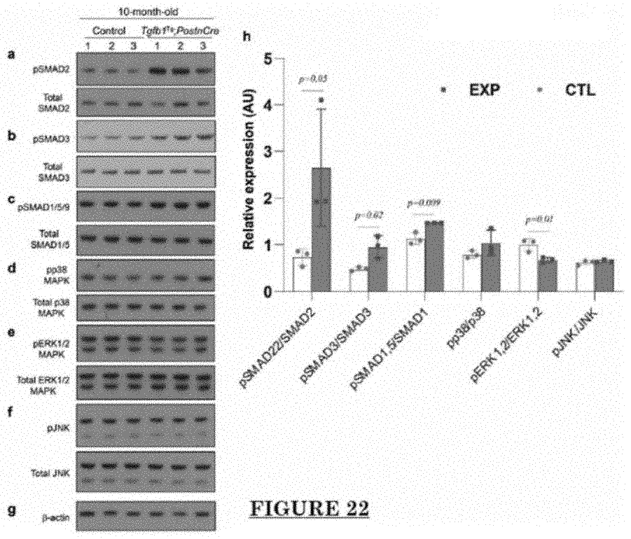

FIG. 22—TGFβ1 hyperactivation in valve interstitial cells leads to increased activation of SMAD-dependent signaling but results in dysregulated activation of components of non-canonical MAPK-dependent TGFβ signaling pathways. α-g, Western blots of 10-month-old male control (n=3) and male Tgƒb1^{TG};PostnCre (n=3) mice showing changes of phosphorylated and total proteins in canonical (pSMAD2/SMAD2, pSMAD3/SMAD3, pSMAD1/5/SMAD1/5) and non-canonical (pp38 MAPK/p38MAPK pERK1/2 MAPK/ERK1/2 MAPK, pJNK/JNK) TGFβ signaling pathways in microdissected AV tissue. β-actin was used as a loading control. Data are mean±s.e.m. P-values and individual values are indicated. Unpaired Two-sided Student's t-tests (without corrections for multiple comparisons) were used.

```
GENETIC SEQUENCES-SEQUENCE LISTINGS
Transforming growth factor beta 1 [Sus scrofa)
GenBank: AAL57902.1
Normal Protein Sequence: 1-390 amino acid
<210> 1
<211> 390
<212> PRT
<213> Susscrofa
<400> 1
Met Pro Pro Ser Gly Leu Arg Leu Leu Pro Leu Leu Leu Pro Leu Leu Trp Leu Leu Val Leu Thr Pro Gly
1 5 10 15 20
Arg Pro Ala Ala Gly Leu Ser Thr Cys Lys Thr Ile Asp Met Glu Leu Val Lys Arg Lys Arg Ile Glu Ala Ile
25 30 35 40 45
Arg Gly Gln Ile Leu Ser Lys Leu Arg Leu Ala Ser Pro Pro Ser Gln Gly Asp Val Pro Pro Gly Pro Leu Pro
50 55 60 65 70
Glu Ala Val Leu Ala Leu Tyr Asn Ser Thr Arg Asp Arg Val Ala Gly Glu Ser Val Glu Pro Glu Pro Glu Pro
75 80 85 90 95
Glu Ala Asp Tyr Tyr Ala Lys Glu Val Thr Arg Val Leu Met Val Glu Ser Gly Asn Gln Ile Tyr Asp Lys Phe
100 105 110 115 120
Lys Gly Thr Pro His Ser Leu Tyr Met Leu Phe Asn Thr Ser Glu Leu Arg Glu Ala Val Pro Glu Pro Val
125 130 135 140 145
Leu Leu Ser Arg Ala Glu Leu Arg Leu Leu Arg Leu Lys Leu Lys Val Glu Gln His Val Glu Leu Tyr Gln
        150 155 160 165 170
Lys Tyr Ser Asn Asp Ser Trp Arg Tyr Leu Ser Asn Arg Leu Leu Ala Pro Ser Asp Ser Pro Glu Trp Leu
                175 180 185 190 195
Ser Phe Asp Val Thr Gly Val Val Arg Gln Trp Leu Thr Arg Arg Glu Ala Ile Glu Gly Phe Arg Leu Ser Ala
                        200 205 210 215 220
His Cys Ser Cys Asp Ser Lys Asp Asn Thr Leu His Val Glu Ile Asn Gly Phe Asn Ser Gly Arg Arg Gly
                        225 230 235 240 245
Asp Leu Ala Thr Ile His Gly Met Asn Arg Pro Phe Leu Leu Leu Met Ala Thr Pro Leu Glu Arg Ala Gln
                                  250 255 260 265
His Leu His Ser Ser Arg His Arg Arg Ala Leu Asp Thr Asn Tyr Cys Phe Ser Ser Thr Glu Lys Asn Cys
270 275 280 285 290
Cys Val Arg Gln Leu Tyr Ile Asp Phe Arg Lys Asp Leu Gly Trp Lys Trp Ile His Glu Pro Lys Gly Tyr His
        295 300 305 310 315
Ala Asn Phe Cys Leu Gly Pro Cys Pro Tyr Ile Trp Ser Leu Asp Thr Gln Tyr Ser Lys Val Leu Ala Leu Tyr
        320 325 330 335 340
Asn Gln His Asn Pro Gly Ala Ser Ala Ala Pro Cys Cys Val Pro Gln Ala Leu Glu Pro Leu Pro Ile Val Tyr
        345 350 355 360 365
Tyr Val Gly Arg Lys Pro Lys Val Glu Gln Leu Ser Asn Met Ile Val Arg Ser Cys Lys Cys Ser
        370 375 380 385 390
Transforming growth factor beta 1 [Sus scrofa)
Transgenic (Modified) Protein Sequence: 1-402 amino acid
<210> 2
<211> 402
<212> PRT
<213> Sus scroja
<400> 2
Met Pro Pro Ser Gly Leu Arg Leu Leu Pro Leu Leu Leu Pro Leu Leu Trp Leu Leu Val Leu Thr Pro Gly
1 5 10 15 20
Arg Pro Ala Ala Gly Leu Ser Thr Cys Lys Thr Ile Asp Met Glu Leu Val Lys Arg Lys Arg Ile Glu Ala Ile
25 30 35 40 45
Arg Gly Gln Ile Leu Ser Lys Leu Arg Leu Ala Ser Pro Pro Ser Gln Gly Asp Val Pro Pro Gly Pro Leu Pro
50 55 60 65 70
Glu Ala Val Leu Ala Leu Tyr Asn Ser Thr Arg Asp Arg Val Ala Gly Glu Ser Val Glu Pro Glu Pro Glu Pro
75 80 85 90 95
Glu Ala Asp Tyr Tyr Ala Lys Glu Val Thr Arg Val Leu Met Val Glu Ser Gly Asn Gln Ile Tyr Asp Lys Phe
100 105 110 115 120
Lys Gly Thr Pro His Ser Leu Tyr Met Leu Phe Asn Thr Ser Glu Leu Arg Glu Ala Val Pro Glu Pro Val
125 130 135 140 145
Leu Leu Ser Arg Ala Glu Leu Arg Leu Leu Arg Leu Lys Leu Lys Val Glu Gln His Val Glu Leu Tyr Gln
        150 155 160 165 170
Lys Tyr Ser Asn Asp Ser Trp Arg Tyr Leu Ser Asn Arg Leu Leu Ala Pro Ser Asp Ser Pro Glu Trp Leu
                175 180 185 190 195
Ser Phe Asp Val Thr Gly Val Val Arg Gln Trp Leu Thr Arg Arg Glu Ala Ile Glu Gly Phe Arg Leu Ser Ala
                        200 205 210 215 220
His Ser Ser Ser Asp Ser Lys Asp Asn Thr Leu His Val Glu Ile Asn Gly Phe Asn Ser Gly Arg Arg Gly
                        225 230 235 240 245
Asp Leu Ala Thr Ile His Gly Met Asn Arg Pro Phe Leu Leu Leu Met Ala Thr Pro Leu Glu Arg Ala Gln
                                250 255 260 265
His Leu His Ser Ser Arg His Arg Arg Ala Leu Asp Thr Asn Ser Tyr Prs Tyr Asp Vas Pro Asp Tyr AG
270 275 280 285 290
Ser Les Tyr Cys Phe Ser Ser Thr Glu Lys Asn Cys Cys Val Arg Gln Leu Tyr Ile Asp Phe Arg Lys Asp
        295 300 305 310 315
Leu Gly Trp Lys Trp Ile His Glu Pro Lys Gly Tyr His Ala Asn Phe Cys Leu Gly Pro Cys Pro Tyr Ile Trp
        320 325 330 335 340
Ser Leu Asp Thr Gln Tyr Ser Lys Val Leu Ala Leu Tyr Asn Gln His Asn Pro Gly Ala Ser Ala Ala Pro
        345 350 355 360 365
Cys Cys Val Pro Gln Ala Leu Glu Pro Leu Pro Ile Val Tyr Tyr Val Gly Arg Lys Pro Lys Val Gi Gln Leu
        370 375 380 385 390
Ser Asn Met Ile Val Arg Ser Cys Lys Cys Ser
        395 400
```

See, Wolfraim L A, Alkemade G M, Alex B, Sharpe S, Parks W T and Letterio J J. Development and application of fully functional epitope-tagged forms of transforming growth factor-beta. J Immunol Methods 2002; 266:7-18. This reference described the HA-Tagged TGFβ1 cDNA sequence in FIG. 11C. Hall BE, Zheng C, Swaim W D, Cho A, Nagineni C N, Eckhaus M A, Flanders K C, Ambudkar I S, Baum B J and Kulkarni A B. Conditional overexpression of TGF-beta1 disrupts mouse salivary gland development and function. Lab Invest. 2010; 90:543-555.

This reference used the HA-Tagged TGFβ1 cDNA and made the Tgƒb1 transgenic mice that we used to create valve interstitial cell-specific TGFβ1 transgenic mice, which develop calcific aortic valve disease All patents, patent applications, published applications, and publications, databases, websites and other published materials cited herein are hereby incorporated by reference to the same extent as though each individual publication, published patent document, or patent application was specifically and individually indicated as being incorporated by reference.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein.

What is claimed is:

1. A mouse model comprising:
at least one transgenic Tgƒb1^{TG};PostnCre mouse bred from a Tgƒb1^{Tg} female mouse and a Peri CreTg,+male mouse; and
wherein the at least one transgenic Tgƒb1^{TG};PostnCre mouse overexpresses bioactive TGFβ1.

2. The mouse model of claim 1, wherein the mouse model does not require any additional experimental insults including hyperlipidemia, hypercholesterolemia, hypertension, diabetes and/or dietary supplements for inducing carotid artery stenosis.

3. The mouse model of claim 1, wherein Tgƒb1 cDNA of the Tgƒb1^{TG};PostnCre mouse has mutated to prevent assembly of at least one latent associated peptide.

4. The mouse model of claim 1, wherein the at least one transgenic Tgƒb1^{TG};PostnCre mouse is born with three aortic valve leaflets wherein right and left coronary cusps of an aortic valve fuse at a base and form a raphe as the at least one Tgƒb1^{TG};PostnCre mouse grows.

5. The mouse model of claim 4, wherein fusing of the right and left coronary cusps is an acquired phenotype and not a congenital malformation.

6. The mouse model of claim 1, wherein mouse genetic sequence 1 is changed to mouse genetic sequence 2.

7. The mouse model of claim 1, wherein calcific AS caused by GFβ1 hyperactivation predominantly in the at least one transgenic Tgƒb1T^{TG};PostnCre mouse is mediated by increased activation of SMAD2, SMAD3, SMAD1/5/9 and p38 MAPK; and decreased activation of pERK1/2 MAPK signaling pathways.

8. The mouse model of claim 1, wherein exposing the at least one transgenic Tgƒb1^{TG};PostnCre mouse to TGFβ receptor I kinase (SB431542) inhibitor attenuates initiation of CAVD in utero.

9. The mouse model of claim 1, wherein carotid artery stenosis shows sex-specific disparity with male transgenic Tgƒb1^{TG};PostnCre mice developing carotid artery stenosis.

10. The mouse model of claim 1, wherein carotid artery stenosis is asymmetric in initiation and progression of calcification in the at least one transgenic Tgƒb1^{TG};PostnCre mouse.

11. A method for creating a mouse model comprising:
mating at least one Tgƒb1^{Tg} female mouse with at least one Peri CreTg,+male mouse; and
producing at least one transgenic Tgƒb1^{TG};PostnCre mouse that overexpresses bioactive TGFβ1.

12. The method of claim 11 further comprising mating at least one Tgƒb1+/−female mouse to a Tgƒb1^{Tg},+; Peri CreTg,+male mouse to produce at least one Tgƒb1^{TG}; +; Peri CreTg,+;Tgƒb1+/−mouse.

13. The method of claim 11 wherein the method does not require any additional experimental insults including hyperlipidemia, hypercholesterolemia, hypertension, diabetes and/or dietary supplements for inducing carotid artery stenosis in the at least one transgenic Tgƒb1^{TG};PostnCre mouse.

14. The method of claim 11, further comprising mutating Tgƒb1 cDNA of the Tgƒb1^{TG};PostnCre mouse to prevent assembly of at least one latent associated peptide.

15. The method of claim 11, further comprising producing at least one male transgenic Tgƒb1^{TG};PostnCre mouse with three aortic valve leaflets wherein right and left coronary cusps of an aortic valve fuse at a base and form a raphe as the at least one male Tgƒb1^{TG};PostnCre mouse grows.

16. The method of claim 15, further comprising wherein fusing of the right and left coronary cusps is an acquired phenotype and not a congenital malformation.

17. The method of claim 11, further comprising mutating mouse genetic sequence 1 to mouse genetic sequence 2 via changing Cysteine at 223 and 225 to Serine.

18. The method of claim 11, further comprising causing calcific AS by GFβ1 hyperactivation predominantly in the at least one transgenic Tgƒb1^{TG};PostnCre mouse mediated by increased activation of SMAD2, SMAD3, SMAD1/5/9 and p38 MAPK; and decreased activation of pERK1/2 MAPK signaling pathways.

19. The method of claim 11, further comprising exposing at least one transgenic Tgƒb1^{TG};PostnCre mouse to TGFβ receptor I kinase (SB431542) inhibitor in utero to attenuate initiation of CAVD.

20. The method of claim 11, further comprising carotid artery stenosis showing sex-specific disparity with male transgenic Tgƒb1^{TG};PostnCre mice developing CAS.


SEQUENCE LISTING (2 sequences)

SEQ ID NO: 1 - protein, 390 aa, Sus scrofa
MPPSGLALLPLLLPLLWLLVLTPGRPAAGLSTCKTIDMELVKRKRIEAIRGQILSKLRLASPPSQGDVPPGPLPEAVLALYNSTRDRVAGESVEPEPEPEADYYAKEVTRVLMVESGNQIYDKFKGTPHSLYMLFNTSELREAVPEPVLLSRAELRLLRLKLKVEQHVELYQKYSNDSWRYLSNRLLAPSDSPEWLSFDVTGVVRQWLTRREAIEGFRLSAHCSCDSKDNTLHVEINGFNSGRRGDLATIHGMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 402 aa, Sus scrofa
MPPSGLRLLPLLLPLLWLLVLTPGRPAAGLSTCKTIDMELVKRKRIEAIRGQILSKLRLASPPSQGDVPPGPLPEAVLALYNSTRDRVAGESVEPEPEPEADYYAKEVTRVLMVESGNQIYDKFKGTPHSLYMLFNTSELREAVPEPVLLSRAELRLLRLKLKVEQHVELYQKYSNDSWRYLSNRLLAPSDSPEWLSFDVTGVVRQWLTRREAIEGFRLSAHSSSDSKDNTLHVEINGFNSGRRGDLATIHGMNRPFLLLMATPLERAQHLHSSRHRRALDTNSYPYDVPDYASLYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS